(19) United States
(12) Patent Application Publication
(10) Pub. No.: US 2025/0052985 A1
(43) Pub. Date: Feb. 13, 2025

(54) SYSTEM AND METHOD FOR QUANTITATIVE PHASE IMAGING FOR MULTI-PARAMETRIC SCREENING OF TUMOR THERAPY RESPONSE

(71) Applicant: UNIVERSITY OF UTAH RESEARCH FOUNDATION, Salt Lake City, UT (US)

(72) Inventors: Thomas A. Zangle, Salt Lake City, UT (US); Philip S. Bernard, Salt Lake City, UT (US); Edward Polanco, Salt Lake City, UT (US); Tarek Moustafa, Salt Lake City, UT (US)

(21) Appl. No.: 18/797,238

(22) Filed: Aug. 7, 2024

Related U.S. Application Data
(60) Provisional application No. 63/517,976, filed on Aug. 7, 2023.

(51) Int. Cl. G02B 21/00; G01N 33/50

(52) U.S. Cl. CPC G02B 21/0028; G01N 33/5011; G02B 21/0032; G02B 21/0036; G02B 21/0056; G02B 21/008

(57) ABSTRACT

A microscope imaging system including a microscope having a stage with a well plate having a plurality of therapy treatment samples, an array of light emitting diodes (LEDs) to illuminate the plurality of therapy treatment samples, and a camera to capture images of the plurality of therapy treatment samples over a period of time using the array of LEDs. The microscope imaging system including an electronic controller configured to receive a plurality of images of the plurality of therapy treatment samples over the period of time from the camera, determine a cell mass for each therapy treatment sample based on each image, track the cell mass over the period of time for each therapy treatment sample, determine a plurality of response parameters for each therapy treatment sample based on the tracked cell mass, and determine a treatment response for each therapy treatment sample based on the plurality of response parameters.

### CROSS-REFERENCE TO RELATED APPLICATIONS

This application is a non-provisional of and claims the benefit of U.S. Provisional Application No. 63/517,976, filed on Aug. 7, 2023, the entire contents of which are incorporated herein by reference.

### STATEMENT REGARDING FEDERALLY SPONSORED RESEARCH OR DEVELOPMENT

This invention was made with government support under W81XWH-19-1-0065 and W81XWH-19-1-0066 awarded by the USAMRAA. The government has certain rights in the invention.

### FIELD OF THE DISCLOSURE

The present disclosure is directed to a quantitative phase imaging system for determining multiple response parameters of therapeutic treatments.

### BACKGROUND

Precision oncology can be useful for improving outcomes in cancer patients by tailoring effective therapies to an individual patient's tumor while minimizing toxic side effects from ineffective drugs. In some implementations, biomarker-driven personalized cancer treatment can improve response rates and extend progression-free survival. Sequencing studies using large oncogene panels in advanced cancers find an actionable DNA mutation in 5-35% of cases, depending on associated tumor histology. Although some mutations are exceptional responders to targeted therapy, rarely do advanced cancer patients with a candidate “targetable” mutation exhibit long-term survival. Thus, there is a need in precision oncology to implement functional cell-based assays to complement genomic panels.

Recent advances in tumor cell expansion allow for the development of ex vivo patient-derived models of cancer that faithfully recapitulate clinical behavior in terms of drug response. Ex vivo testing is also amenable to clinical testing since ex vivo can be multiplexed and completed within weeks of tumor sample collection. Considering the fast turnaround time from tumor sample collection, ex vivo testing also allows many more drugs to be screened at a lower cost, and on a timescale with potential for informing patient care. A variety of analytic methods for measuring the response of cultured cells to drug exposure are presently employed. Cell culture-based drug-screening assays vary from simple cell counts and determination of live: dead ratios with stains, to metabolic assays (e.g., assays for determining the release of ATP or lactate12), to measurement of specific programmed cell death effectors such as caspases or BH3-domain activation. CellTiter-Glo (CTG), for example, is an assay that measures cell ATP content as a proxy for cell viability. When used as an endpoint assay post drug exposure, CTG can produce reproducible drug response data, more rapidly and with less bias than cell counting. CTG can also produce greater signal-to-noise ratio than other luminescence assays such as Toxilight and resazurin-based assays. However, the described measures are typically applied as bulk, endpoint assays, and are incapable of capturing the dynamics of single-cell responses to therapy.

In contrast to endpoint assays, real-time assays can elucidate the temporal dynamics of drug response. In some implementations, real-time assays discriminate between a cytostatic response where cell growth is substantially reduced and a cytotoxic response where the therapy induces cell death. For example, incubator-housed microscope systems for measuring real-time cell proliferation (e.g., Incucyte) can yield results concordant to CTG and BH3 profiling. As a longitudinal imaging approach, the Incucyte measures parameters such as population-averaged proliferation rate and cell viability throughout the experimental duration granting insight into changes in cell behavior throughout the course of imaging. An emerging alternative is to use cell mass accumulation rate as a measure of cell growth. For example, suspended microchannel resonators are a highly sensitive tool for measuring changes in cell mass. Microchannel resonators can measure statistically meaningful changes in cell growth from very short duration (˜10 min) measurements, or individual resonators can be used for longitudinal imaging of cell behavior in response to drugs. However, suspended microchannel resonators are limited by the need to flow cells through individual resonators and work best with non-adherent cell types.

Thus, systems and methods that provide real-time imaging of cell-based assays with capturing dynamic single-cell responses, faster response rate determination, and reduced cost would be desirable.

### SUMMARY

Quantitative phase imaging (QPI) measures the growth rate of individual cells by quantifying changes in mass versus time. In some implementations, breast cancer cell lines MCF-7, BT-474, and MDA-MB-231 can be used to validate QPI as a multiparametric approach for determining response to single-agent therapies. The validation of QPI allows for rapid determination of drug sensitivity, cytotoxicity, heterogeneity, and time of response for about 100,000 individual cells or small clusters in a single experiment. In some examples, QPI half maximal effective concentration (EC₅₀) values are concordant with CTG, a gold standard metabolic endpoint assay. Additionally, by applying multiparametric QPI, cytostatic/cytotoxic and rapid/slow responses can be characterized, and the emergence of resistant subpopulations can be tracked. Thus, QPI reveals dynamic changes in response heterogeneity in addition to average population responses, a key advantage over endpoint viability or metabolic assays. Overall, multiparametric QPI reveals a rich picture of cell growth by capturing the dynamics of single-cell responses to candidate therapies.

In one aspect, the disclosure provides a microscope imaging system including a quantitative phase imaging microscope. The quantitative phase imaging microscope includes a stage configured to hold and move a well plate having a plurality of therapy treatment samples, an array of light emitting diodes (LEDs) configured to illuminate the plurality of therapy treatment samples, and a camera configured to capture images of the plurality of therapy treatment samples over a period of time using an amount of illumination provided by the array of LEDs. The microscope imaging system also includes an electronic controller communicatively coupled to the quantitative phase imaging microscope. The electronic controller is configured to receive a plurality of images of the plurality of therapy treatment samples over the period of time from the camera, determine a cell mass for each therapy treatment sample of the plurality of therapy treatment samples based on each image of the plurality of images, track the cell mass over the period of time for each therapy treatment sample of the plurality of therapy treatment samples, determine a plurality of response parameters for each therapy treatment sample of the plurality of therapy treatment samples based on the cell mass over the period of time, and determine a treatment response for each therapy treatment sample of the plurality of therapy treatment samples based on the plurality of response parameters.

In some aspects, the plurality of response parameters include at least one selected from the group consisting of a specific growth rate, a half maximal effective concentration, a depth of response, a time of response, and a standard deviation of response.

In some aspects, each therapy treatment sample of the plurality of therapy treatment samples includes live cancer cells exposed to a different therapeutic drug.

In some aspects, the cell mass is a mass of the live cancer cells exposed to the different therapeutic drugs.

In some aspects, the quantitative phase imaging microscope further includes a lens configured to focus the camera on the plurality of therapy treatment samples.

In some aspects, when determining the cell mass for each therapy treatment sample of the plurality of therapy treatment samples, the electronic controller is further configured to determine a phase shift of the amount of illumination passing through each therapy treatment sample of the plurality of therapy treatment samples.

In some aspects, when tracking the cell mass over the period of time for each therapy treatment sample of the plurality of therapy treatment samples, the electronic controller is further configured to determine a mass accumulation rate for each therapy treatment sample of the plurality of therapy treatment samples based on the determined cell mass and the period of time.

In another aspect, the disclosure provides a method for determining treatment response parameters with a microscope imaging system. The method includes receiving, by an electronic controller, a plurality of images of a plurality of therapy treatment samples over a period of time from a camera, determining, via the electronic controller, a cell mass for each therapy treatment sample of the plurality of therapy treatment samples based on each image of the plurality of images, tracking, via the electronic controller, the cell mass over the period of time for each therapy treatment sample of the plurality of therapy treatment samples, and determining, via the electronic controller, a plurality of response parameters for each therapy treatment sample of the plurality of therapy treatment samples based on the cell mass over the period of time.

In some aspects, the method includes determining, via the electronic controller, a treatment response for each therapy treatment sample of the plurality of therapy treatment samples based on the plurality of response parameters.

In some aspects, the plurality of response parameters include at least one selected from the group consisting of a specific growth rate, a half maximal effective concentration, a depth of response, a time of response, and a standard deviation of response.

In some aspects, each therapy treatment sample of the plurality of therapy treatment samples includes live cancer cells exposed to a different therapeutic drug.

In some aspects, the cell mass is a mass of the live cancer cells exposed to the different therapeutic drugs.

In some aspects, the method includes determining, via the electronic controller, a phase shift of the amount of illumination passing through each therapy treatment sample of the plurality of therapy treatment samples.

In some aspects, the method includes determining, via the electronic controller, a mass accumulation rate for each therapy treatment sample of the plurality of therapy treatment samples based on the determined cell mass and the period of time.

In another aspect, the disclosure provides a quantitative phase imaging microscope including a camera configured to capture images of a plurality of therapy treatment samples over a period of time and an electronic controller communicatively coupled to the quantitative phase imaging microscope. The electronic controller is configured to receive a plurality of images of the plurality of therapy treatment samples over the period of time from the camera, determine a cell mass for each therapy treatment sample of the plurality of therapy treatment samples based on each image of the plurality of images, track the cell mass over the period of time for each therapy treatment sample of the plurality of therapy treatment samples, and determine a plurality of response parameters for each therapy treatment sample of the plurality of therapy treatment samples based on the cell mass over the period of time.

In some aspects, the electronic controller is configured to determine a treatment response for each therapy treatment sample of the plurality of therapy treatment samples based on the plurality of response parameters.

In some aspects, the electronic controller is configured to determine the plurality of response parameters for each therapy treatment sample of the plurality of therapy treatment samples in cells from a patient derived organoid.

In some aspects, the electronic controller is configured to determine a difference in the plurality of response parameters between each therapy treatment sample of the plurality of treatment samples.

In some aspects, the difference in the plurality of response parameters is determined from a tumor organoid.

In some aspects, the electronic controller is configured to determine a tumor cell from a non-tumor cell for each therapy treatment sample of the plurality of therapy treatment samples based on the cell mass over the period of time.

Other aspects of various embodiments will become apparent by consideration of the detailed description and accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A illustrates an example plate setup for measuring a 6-point dose response of five cancer therapies in triplicate in addition to two solvent controls, a growth response of cells are measured noninvasively using QPI before being assayed using CTG, and a system for measuring the growth response of cells using QPI, according to some embodiments.

FIG. 1B illustrates a timeline showing two plates set up in parallel to measure cell viability, according to some embodiments.

FIG. 1C is an image showing representative QPI data from a single location, according to some embodiments.

FIG. 1D is an image showing representative QPI data from a single location, according to some embodiments.

FIGS. 1E-1F are QPI images of a growing cell in a dimethyl sulfoxide (DMSO) (solvent control) at 1 hour and at 16 hours, according to some embodiments.

FIG. 1G is a graph of a measurement of growing cell mass versus time and other cells from the same well of FIGS. 1E-1F, according to some embodiments.

FIGS. 1H-1I are QPI images of a dying cell in 2 μM doxorubicin shown at 20 hours while still growing and at 60 hours, after the cell death event has occurred, according to some embodiments.

FIG. 1J is a graph of a measurement of dying cell mass versus time and other cells from the same well of FIGS. 1H-1I, according to some embodiments.

FIG. 1K is a graph of QPI-based measured parameters in response to therapy, according to some embodiments.

FIG. 2A is a graph of QPI measurements of the specific growth rate distributions versus doxorubicin concentration, according to some embodiments.

FIG. 2B is a graph of points on a dose response curve representing specific growth rate of cells, according to some embodiments.

FIG. 2C is a graph of average cell mass normalized by initial cell mass of each cell cluster versus time, according to some embodiments.

FIG. 2D is a graph of a comparison of EC₅₀ from Hill equation fitting to CTG and QPI data, according to some embodiments.

FIG. 2E is a confusion matrix of precision and accuracy of QPI relative to CTG, according to some embodiments.

FIG. 3A is a graph of mass versus time normalized by initial cell mass averaged over all BT-474 cell clusters at each time point, according to some embodiments.

FIG. 3B is a graph of mass versus time normalized by the initial cell mass averaged over all cells at each time point, according to some embodiments.

FIG. 3C is a graph of BT-474 response, according to some embodiments.

FIG. 3D is a graph of Hellinger distance versus time for determining probability distributions, according to some embodiments.

FIGS. 3E-3F are graphs of time of response (ToR) near EC₅₀ versus depth of response (DoR) from QPI data, according to some embodiments.

FIG. 3H is a graph of normalized cell mass versus time for cancer cells, according to some embodiments.

FIG. 4A is a graph of growth rate distribution of 21,791 MDA-MB-231 cells at the end of 72 hours of 20 μM of indicated drug exposure, according to some embodiments.

FIG. 4B is a graph of MDA-MB-231 population growth rate distributions during 72 hour treatment with DMSO and 20 μM docetaxel, according to some embodiments.

FIG. 4C is a graph of mass versus time tracks for two indicated cells of FIG. 4B, according to some embodiments.

FIGS. 4D-4E are images of a control cell from a cell panel and a large cell from the cell panel, according to some embodiments.

FIG. 4F is a histogram showing a final mass for docetaxel treated cells and DMSO treated cells, according to some embodiments.

FIG. 4G is a graph of standard deviation at EC₅₀ plotted against EC₅₀ for cell growth, according to some embodiments.

FIG. 4H is a graph of normalized area under precision-recall curve (AUPRC) plotted against a percentage of control cells mixed into a drug-treated population, according to some embodiments.

FIG. 5A is a correlation matrix showing a relationship of functional measurements and predictive of cancer therapies affecting heterogeneity, according to some embodiments.

FIGS. 5B-5C are graphs of EC_{50,SD} correlated with EC_{50,SGR} and standard deviation in growth at 20 μM concentration correlated with standard deviation of growth at EC₅₀, according to some embodiments.

FIG. 5D is a graph of a plot of mean specific growth rate against standard deviation in growth parameterized by time for MDA-MB-231 cells, according to some embodiments.

FIG. 5E is a graph of a plot of mean specific growth rate against standard deviation in growth parameterized by time for BT-474 cells, according to some embodiments.

FIG. 5F is a graph of ToR versus EC₅₀ from QPI data, according to some embodiments.

FIG. 6 shows graphs and an image of calibration data for a QPI microscope, according to some embodiments.

FIG. 7 shows images and graphs of QPI measured drug sensitivity and temporal dynamics of cell clusters, according to some embodiments.

FIG. 8 shows graphs of a linear relationship between cell mass and cell growth, according to some embodiments.

FIG. 9 is a graph showing temporal dynamics of drug response for a single MDA-MB-231 cell during exposure to 2 μM doxorubicin, according to some embodiments.

FIG. 10 shows graphs of dose response measurements for cell lines and drug combinations, according to some embodiments.

FIG. 11 shows graphs of 24 hour QPI data predicted results in 72 hour CTG and QPI experiments, according to some embodiments.

FIG. 12 shows dynamic response plots for MCF7 and MDA-MB-231, according to some embodiments.

FIG. 13 shows graphs of Hellinger distance used to determine the statistical distance between drug treated and control treated groups, according to some embodiments.

FIG. 14 shows graphs of measured Hellinger distance, according to some embodiments.

FIG. 15 shows graphs of changes in intrinsic heterogeneity during treatment being both drug and cell-line dependent, according to some embodiments.

FIG. 16 shows graphs of dose response of growth heterogeneity versus therapy concentration, according to some embodiments.

FIG. 17 is a graph of standard deviation at EC₅₀ plotted against DoR, according to some embodiments.

FIG. 18 shows graphs identifying resistant cells using specific growth rate, according to some embodiments.

FIG. 19 shows graphs of correlations between the drug response parameters measured using QPI and orthogonality between measurements, according to some embodiments.

FIG. 20 shows graphs of individual replicates of time parameterized SD vs SGR, according to some embodiments.

FIG. 21 shows graphs of heterogeneity vs specific growth rate parameterized by time for MDA-MB-231 cells showing increase in heterogeneity in response to therapy, according to some embodiments.

FIG. 22 shows graphs of heterogeneity vs specific growth rate parameterized by concentration for MDA-MB-231 and BT-474 cells showing increase in heterogeneity in response to therapy, according to some embodiments.

FIG. 23 shows graphs of heterogeneity vs specific growth rate parameterized by time for BT-474 cells showing increase in heterogeneity in response to therapy, according to some embodiments.

FIG. 24 shows graphs of multiparametric quantitative phase microscope (QPM) data showing multifaceted response of PDxO models to cancer therapy, according to some embodiments.

FIG. 25 shows graphs of multiparametric QPM data showing differential response in tumor cells from varying site of origin, according to some embodiments.

FIG. 26 shows an image and graphs of drug response characteristics of direct from thaw primary sample, according to some embodiments.

FIG. 27 shows an image and graphs of multiparametric characterization of drug response of primary cells after two-week expansion, according to some embodiments.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

Quantitative phase imaging (QPI) is a real-time, label-free technique for determining growth of individual cells by measuring a phase shift of light as it passes through a transparent sample, such as, for example, a cell. The phase shift of light is directly proportional to cell mass, which increases due to cell growth, such as, for example, during progression through a cell cycle. In some instances, cell mass is a mass of live cancer cells exposed to a therapeutic drug. QPI is a real-time, high throughput tool for measuring growth responses of individual cells to therapy. Previous applications of QPI have utilized microfluidics to study cell response to fluidic shear stress or identify resistant cell populations using up to 20 different QPI-derived features such as, for example, area and shape. However, these and other studies of QPI have narrowly focused on measuring the overall sensitivity or toxicity of potential therapies with only limited studies of a heterogeneity of response. Previous work with CellTiter-Glo (CTG) shows that combining multiple measures of cell response is superior to only measuring drug sensitivity. In some instances, QPI can have a greater impact as a tool for functional medicine by enabling simultaneous measurement of multiple parameters that are indicative of cancer cell response to therapy.

In some instances, QPI can be used as a quantitative multiparametric method to characterize dynamic changes in growth rate, drug sensitivity, drug toxicity, heterogeneity, and ToR (e.g., measured parameters) with sufficient throughput to make QPI suitable for clinical applications. The measured parameters, summarized in Table 1 below, are orthogonal measurements that cannot be derived from traditional measurements, such as mean drug sensitivity alone. In some instances, the measured parameters can also be combined to quantify drug-dependent, dynamic responses of cell populations in terms of time-varying mean and standard deviation of growth rates. Taken together, the measured (e.g., QPI-derived) parameters described herein provide a richer, more complete description of cell response to therapy than conventional drug screening approaches.

TABLE 1
Summary of QPI Drug Response Parameters.

| Name | Abbreviation | Description | Units |
|---|---|---|---|
| Specific | SGR | Exponential growth | h⁻¹ |
| growth | | constant, computed | |
| rate | | as the rate of change | |
| | | of cell mass over | |
| | | time, normalized by | |
| | | cell mass. | |
| Half maximal | EC₅₀ | Therapy | μM |
| effective | | concentration at | |
| concentration | | which cells exhibit | |
| | | 50% of maximum | |
| | | response. | |
| Depth | DoR | Maximum difference | |
| of | | in SGR between cells | |
| response | | at minimum and | |
| | | maximum therapy | |
| | | concentrations, | |
| | | computed from Hill | |
| | | sigmoid curve fitting | |
| | | parameters. | |
| Time | ToR | The average time | h |
| of | | required to elicit a | |
| response | | response to therapy at | |
| | | the tested | |
| | | concentration. | |
| Standard | SD | Standard deviations | h⁻¹ |
| deviation | | of SGR at the tested | |
| of | | concentration as a | |
| response | | measure of cell-to- | |
| | | cell heterogeneity. | |

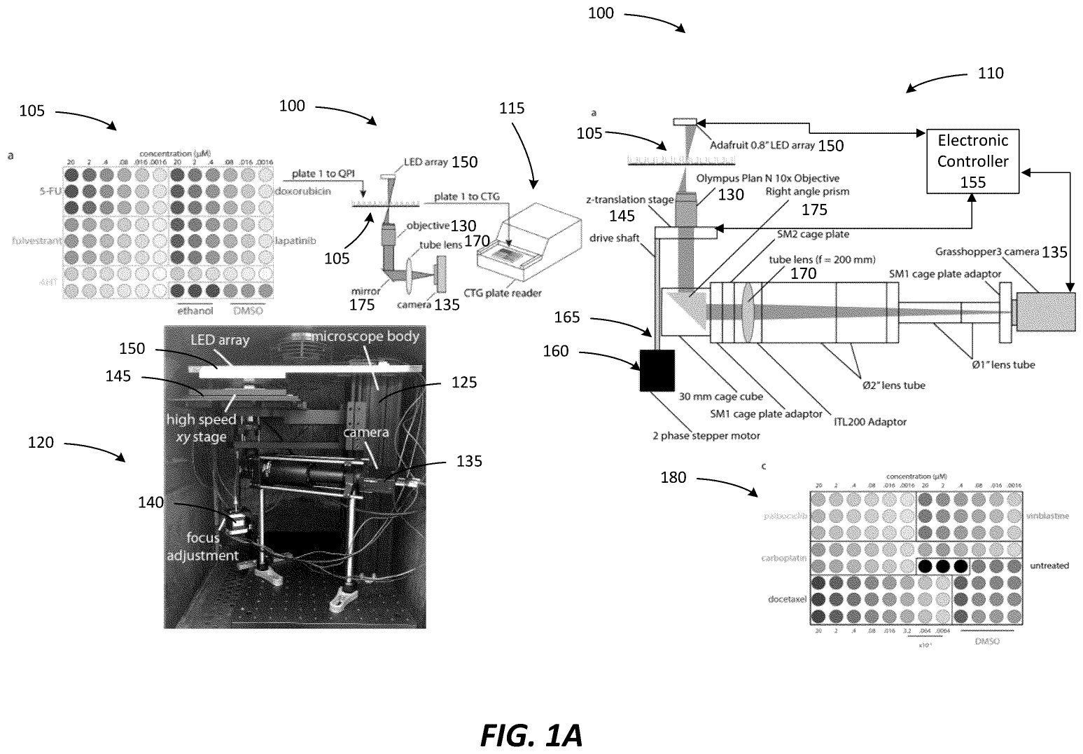

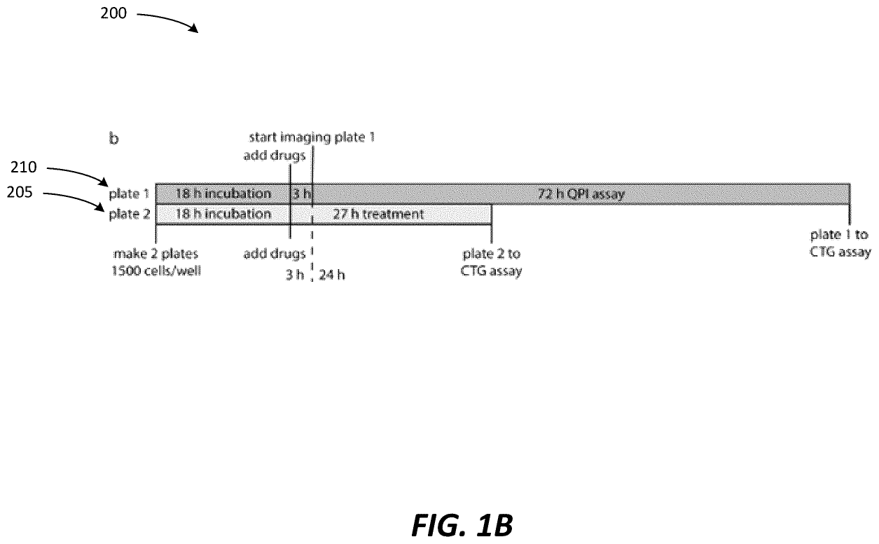

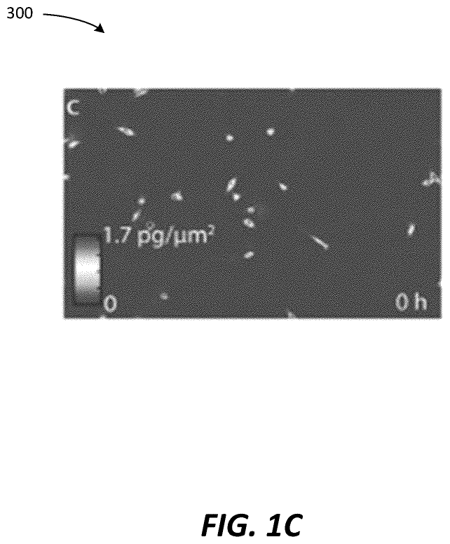

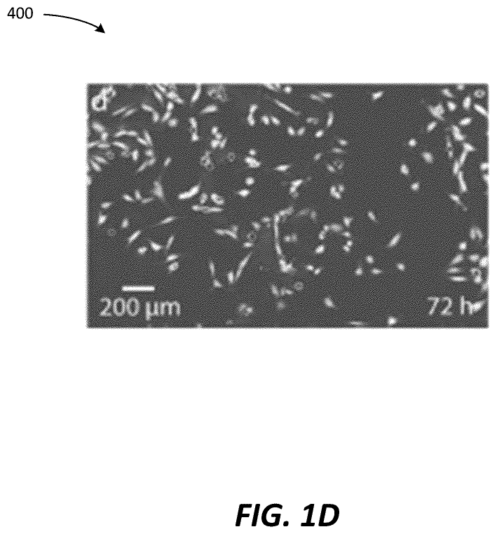

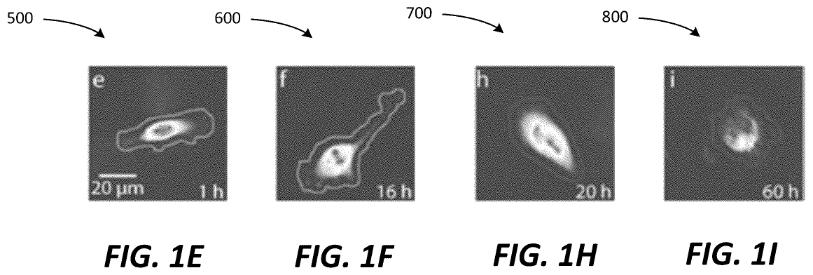

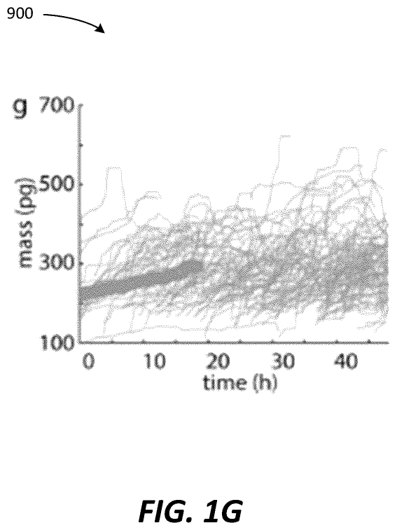

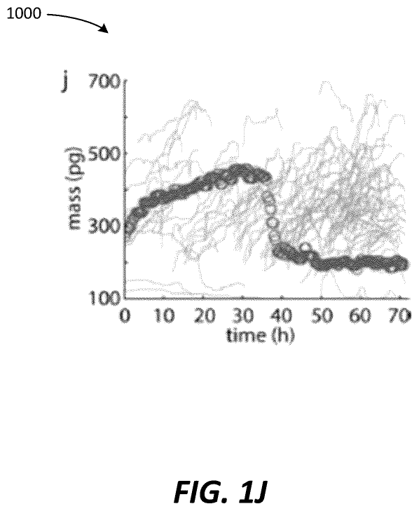

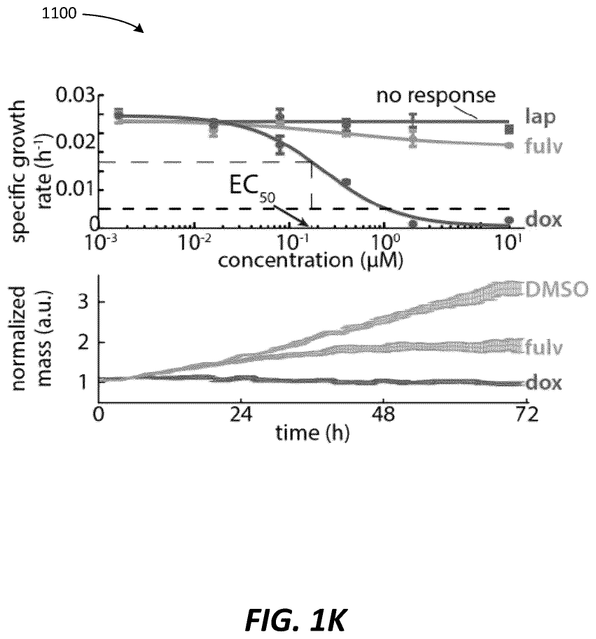

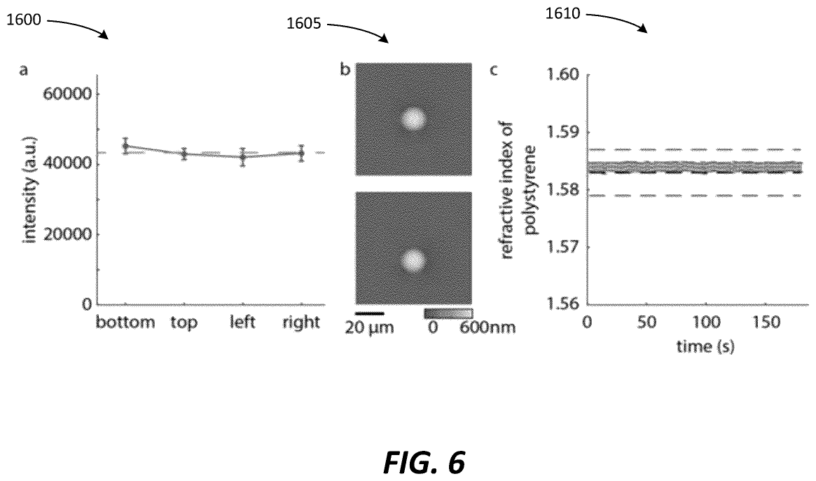

In some embodiments, breast cancer cell lines are imaged, representing diverse clinical subtypes, summarized in Table 2 below, using a QPI microscope (FIG. 1A) based on differential phase contrast (DPC) microscopy and phase reconstruction via a QPI microscope system. DPC is used for image acquisition and phase reconstruction because DPC's flexible design may be customized for high throughput phase measurements. Additionally, in some instances, the compact design of the QPI microscope system fits inside a tissue culture incubator and includes inexpensive optical components that are readily available, as summarized in Table 3 below, facilitating widespread implementation as a clinical screening tool. Before imaging, a QPI microscope system alignment is calibrated, and accuracy and precision are confirmed by measuring a refractive index of polystyrene microbeads (FIG. 6). As illustrated in the embodiment of FIG. 1A, a QPI microscope system 100 conducts assays in a 96-well plate (e.g., a well-plate 105) with 6-point dose response curves and up to five therapies in triplicate including solvent controls, summarized in Table 4 below. As described below, in reference to FIG. 1A, a growth response of cells is measured noninvasively using QPI via a QPI microscope system 110 before being assayed using a CTG reader 115.

TABLE 2
Summary of Cell
Lines and Receptor Status.

| Cell line | Receptor status |
|---|---|
| MCF-7 | ER+/PR+/HER2− |
| MDA-MB-231 | ER−/PR−/HER2− |
| BT-474 | ER−/PR+/HER2+ |

TABLE 3
List of QPI Microscope Components.

| Part | Supplier | Model/part number |
|---|---|---|
| Arduino Metro M4 | Adafruit | 3382 |
| 0.8″, 8 × 8 LED array | Adafruit | 870 |
| High speed xy stage | Thorlabs | MLS203 |
| 10 × Olympus PLAN Objective | Thorlabs | RMS10X |
| z-translation stage | Thorlabs | SM1Z |
| Flexible drive shaft | McMaster-Carr | 3135K15 |
| Sparkfun 2-phase stepper motor | Mouser | 474-ROB-10846 |
| 25 mm right angle prism | Thorlabs | PS911 |
| 30 mm cage cube | Thorlabs | CCM1-4ER |
| SM1 cage plate adaptor | Thorlabs | LCP6X |
| SM2 cage plate | Thorlabs | LCP01 |
| Tube lens (f = 200 mm) | Thorlabs | ITL200 |
| ITL200 adaptor | Thorlabs | SM2A20 |
| Ø2″ lens tube | Thorlabs | SM2L2 |
| Ø1″ lens tube | Thorlabs | SM1L2 |
| Grasshopper3 camera | Teledyne-FLIR | GS3-U3-23S6M-C |

List of Compounds for Solvent Controls.

| Drug name | Abbreviation | Target |
|---|---|---|
| 5-Fluorouracil | 5FU | DNA-RNA synthesis |
| Carboplatin | carb | DNA synthesis |
| Docetaxel | doc | Microtubule |
| Doxorubicin | dox | DNA topoisomerase II |
| Fulvestrant | fulv | Estrogen receptor |
| Lapatinib | lap | EGRF/HER2 |
| Palbociclib | palb | CDK4/6 |
| 4-hydroxy-Tamoxifen | 4HT | Estrogen receptor |
| Vinblastine | vin | Microtubule |

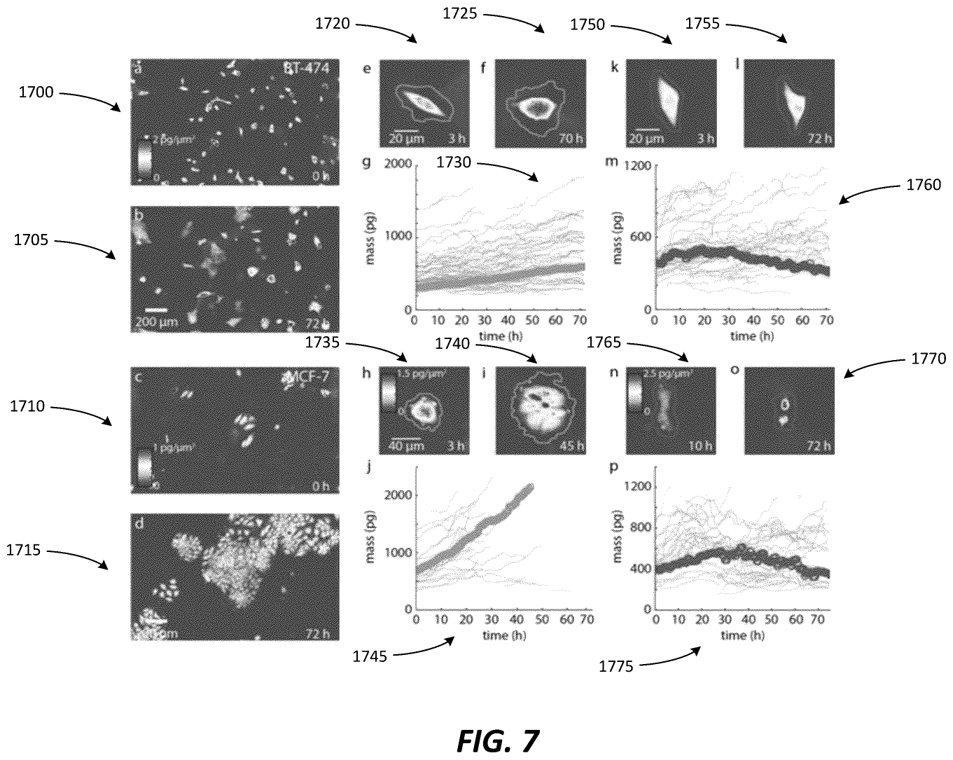

FIG. 1B illustrates a timeline plot 200 showing two plates (e.g., two, separate well-plates 105) set up in parallel to measure cell viability. With reference to FIG. 1B, two plates are set-up simultaneously for each experiment. A first plate 205 is incubated for 27 hours (h) post drug exposure prior to CTG analysis (3 h equilibration+24 h of imaging), which measures cell adenosine triphosphate (ATP) content as a surrogate for cell viability. A second plate 210 is imaged for 72 h after 3 h equilibration (e.g., 75 h) and followed by CTG. As illustrated in FIGS. 1C and 1D, for an example QPI implementation, nine imaging locations are chosen in each well with a minimum of 10 cells or small cell clusters per location. The imaging locations are imaged for 72 h, as shown by images 1700-1715 of FIG. 7.

The image 1700 shows a field of view of BT-474 cells treated with DMSO at the beginning of a 72 h experiment. The image 1705 shows a field of view of BT-474 cells at the end of the experiment where the cells have grown into clusters. The image 1710 shows an example of a well of the well-plate 105 containing MCF-7 cells at the beginning of the experiment from the DMSO control. The image 1715 shows at the end of the experiment, the MCF-7 cells have grown to fill most of the field of view. For example, FIG. 1C is an image 300 showing representative QPI data from a single location within a single DMSO control well at 0 h. FIG. 1D is an image 400 showing representative QPI data from a single location at 72 h with 10-100 MDA-MB-231 cells imaged per location. The scalebar illustrated in FIGS. 1C-1D is 200 μm. In some embodiments, across all 864 possible imaging locations, the growth response of 20,000 to 130,000 cells or cell clusters is measured per 72 h experiment. Cells/clusters are automatically segmented from background for individual quantification of cell mass differentiating healthy growing cells, as illustrated in FIGS. 1E-1G and shown by images and graphs 1720-1745 of FIG. 7, from arrested or dying cells, as illustrated in FIGS. 1H-1J and shown by images and graphs 1750-1775.

The image 1720 shows a cluster of 3 BT-474 cells from the DMSO solvent control that grow into a larger cluster of 6 cells, as shown by the image 1725, that are segmented together (green) to measure the growth of the cluster. The graph 1730 shows individual mass measurements for the cluster of images 1720 and 1725 and are plotted showing robust growth. The image 1735 shows a small cluster of MCF-7 cells treated with DMSO at the beginning of the experiment, grows into a larger cluster by the 45 h timepoint as shown in the image 1740. The graph 1745 shows measuring the mass of the cluster of images 1735 and 1740 demonstrating robust growth. The image 1750 is an example of a small cluster of BT-474 cells exposed to 2 μM of doxorubicin at the beginning of the experiment. At the end of the experiment, the image 1755 shows the cluster has not grown noticeably in size. The graph 1760 shows the clusters mass of images 1750 and 1755 is tracked over time showing the cluster is losing mass. The image 1765 shows MCF-7 cells exposed to 20 μM fulvestrant at the 10 h timepoint and the image 1770 shows MCF-7 cells exposed to 20 μM fulvestrant at the 72 h timepoint appear to shrink in size over the course of the experiment. The graph 1775 shows plotting the mass over time for this small cluster of the images 1765 and 1770, that they grow slowly at the beginning and end the experiment with a lower mass than they started with.

For example, FIG. 1E illustrates a QPI image 500 of a growing cell in a DMSO at 1 h. FIG. 1F illustrates a QPI image 600 of a growing cell in a DMSO at 16 h. FIG. 1H illustrates a QPI image 700 of a dying cell in 2 μM doxorubicin shown at 20 h. FIG. 1I illustrates a QPI image 800 of a dying cell in 2 μM doxorubicin while still growing and at 60 h, after the cell death event has occurred.

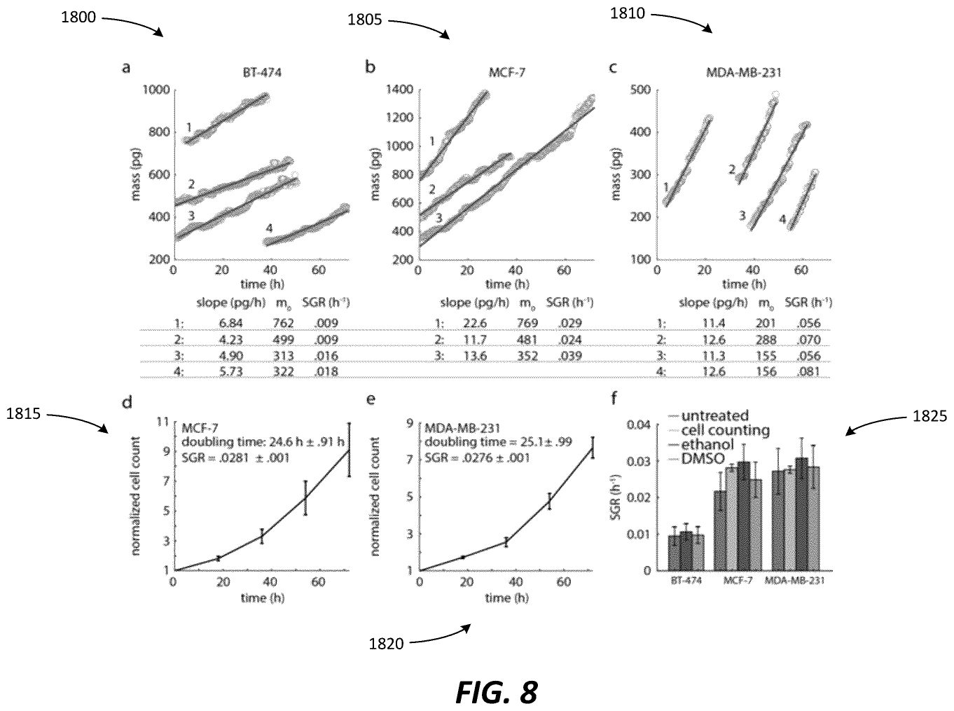

In some embodiments, QPI mass versus time data includes several features that underlie the ability of QPI to distinguish multiple dynamic characteristics of cell responses to drugs. First, the rate of mass accumulation, or cell growth rate (dm/dt), may be used to characterize cell growth. As illustrated in FIG. 1G, in healthy cells, the growth rate is constant as cells accumulate mass during each cell cycle in the DMSO control, as shown by the graphs 1730 and 1745. FIG. 1G is a graph 900 of a measurement of growing cell mass versus time and other cells from the well-plate 105 of, for example, FIGS. 1E-IF. In FIG. 1G, an automatically segmented boundary for healthy growing cell is shown in green. Cell growth rate is typically proportional to the mass of the cell or cluster. A slope of a linear regression for individual cell/cluster mass versus time by the cell/cluster initial mass may be normalized to find the SGR. The SGR may account for variations in growth rate due to differences in cell or cluster size as shown in graphs 1800-1810 of FIG. 8. For example, the graph 1800 shows a mass over time plot for BT-474 cells for a linear fit to measure the growth rate of individual cells. The graph 1805 shows a mass over time plot for MCF-7 cells for a linear fit to measure the growth rate of individual cells. The graph 1810 shows a mass over time plot for MDA-MB-231 cells for a linear fit to measure the growth rate of individual cells.

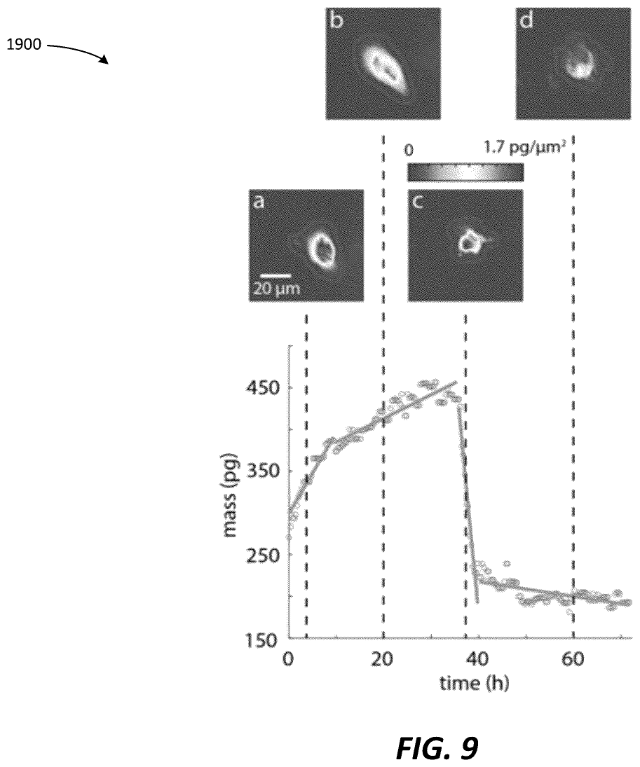

For proliferating cells, the SGR matches the exponential growth constant measured by cell counting as cells double their mass with each cell cycle, as shown by graphs 1815-1825 of FIG. 8. For example, the graph 1815 shows proliferation measurements of MCF-7 cells treated with ethanol showing a doubling time of approximately 24.6 h, which corresponds to an exponential growth constant of 0.0281 h⁻¹. The graph 1820 shows proliferation measurements of ethanol treated MDA-MB-231 cells showing a doubling time of 25.1 h, which corresponds to an exponential growth constant of 0.0276 h⁻¹. The graph 1825 shows growth rate measured using cell counting showing similar results to growth rate measured using QPI. Error bars show the standard error of the mean. However, when exposed to therapies, mass versus time tracks of individual cells or clusters reveal complex dynamic responses. For example, as illustrated in FIGS. 1H-1J, one individual MDA-MB-231 cell exposed to 2 μM doxorubicin exhibits robust growth (SGR=0.0358 h⁻¹, tD=19 h) for the first 10 h before showing reduced growth (SGR=0.0105 h⁻¹, tD=66 h) during the next 26 h, followed by an abrupt decrease in mass during cell death (SGR=−0.217 h⁻¹) and a subsequent gradual loss of mass (SGR=−0.0033 h⁻¹), as illustrated by FIG. 9. FIG. 9 is a graph 1900 showing a point A where a cell initially exhibits robust growth (SGR=0.0358 h−1). The graph 1900 shows a point B after about 13 h of imaging the growth rate of the cell abruptly declines (SGR=0.0105 h−1) for the following 19 h. The graph 1900 shows a point C where the cell then rapidly loses mass (SGR=−0.217 h−1) at approximately 40 h post-exposure during the active stages of cell death. The graph 1900 also shows a point D where the cell then continues losing mass (SGR=−0.0033 h−1) throughout the rest of the experiment.

FIG. 1J is a graph 1000 of a measurement of dying cell mass versus time and other cells from the well-plate 105 of, for example, FIGS. 1H-1I. The dynamic responses are highly heterogeneous from cell to cell or cluster to cluster, as illustrated by the image 600 of FIG. 1F compared to the graph 1000 of FIG. 1J, as shown by the image 1735. As further illustrated by FIG. 1J, an automatically segmented boundary for dying cell is shown in red. A broad range of dynamic behaviors may be captured by label-free, single-cell QPI data during drug response measurements using the QPI microscope system 110. FIG. 1K is a graph 1100 of QPI-based measured parameters in response to therapy using the QPI microscope system 110. For example, the graph 1100 of FIG. 1K illustrates specific growth rate versus concentration and normalized mass versus time for cancer cells in response to different therapeutic treatments (e.g., therapeutic drugs).

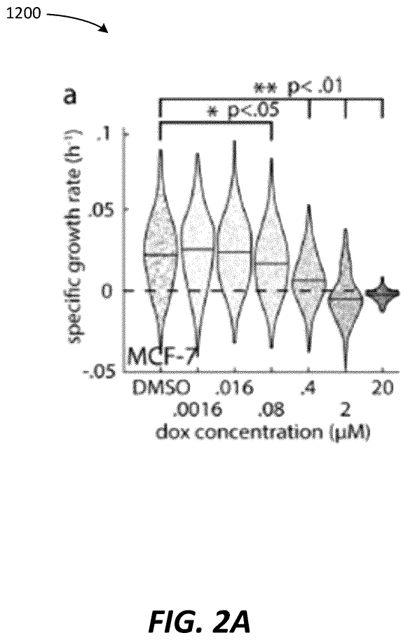

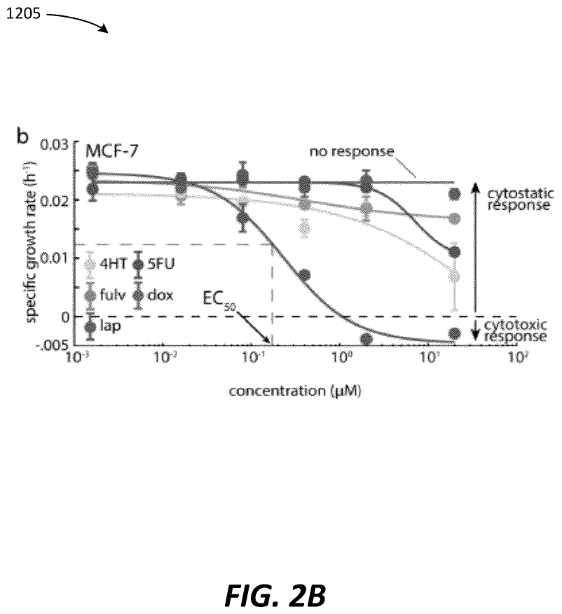

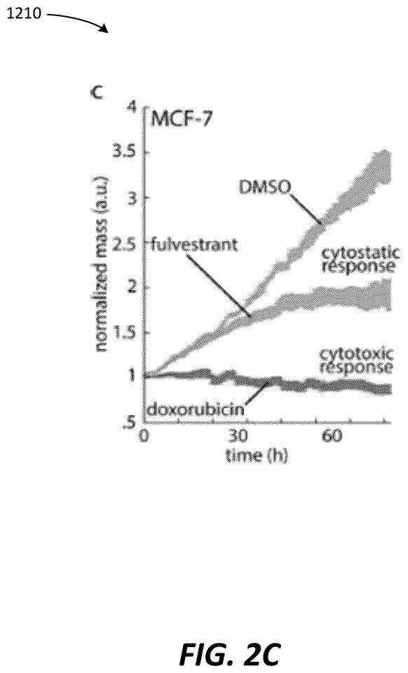

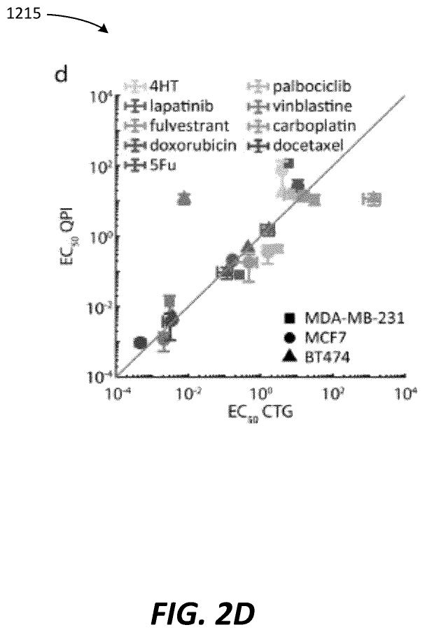

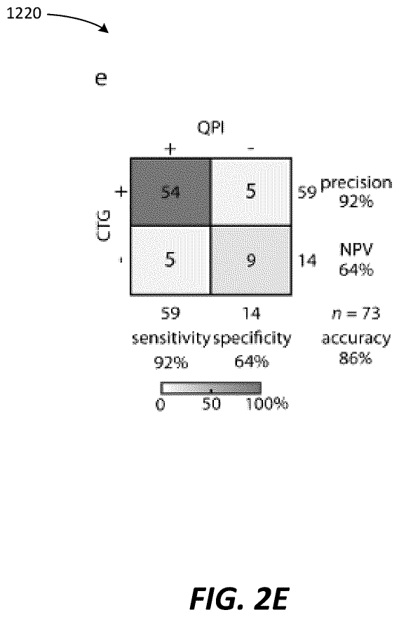

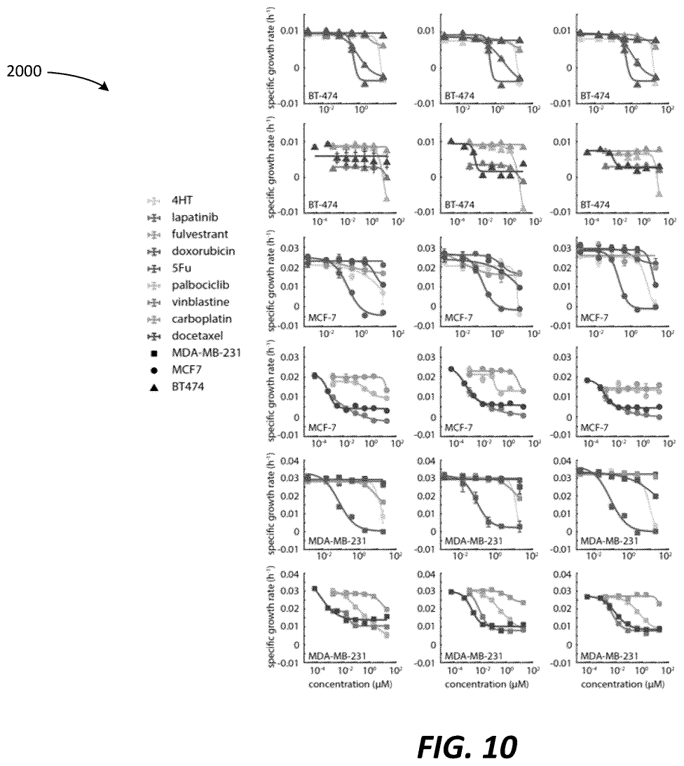

In some embodiments, a dose-dependent change in growth, as indicated by a change in the rate of mass accumulation or loss, is a parameter that can be extracted from QPI dose-response data. For example, FIG. 2A is a graph 1200 showing the SGR distribution for MCF-7 (e.g., Michigan Cancer Foundation-7) cells decreases with increasing doxorubicin concentration to a minimum of −0.003+0.004 h⁻¹ (mean±standard deviation, SD) at 20 μM, which in some instances is the highest dose tested, indicating cellular response to the doxorubicin. Individual specific growth rate measurements are indicated by points in the distribution of the graph 1200 of FIG. 2A. Blue is indicative of the DMSO control and red is indicative of doxorubicin. The black outline is a kernel density function fit to the distribution of specific growth rates for each condition (e.g., *p<0.05, **p<0.01). In some embodiments, the average SGR of each condition is fit to a sigmoid dose-response curve using a Hill equation. For example, computed logistic fit parameters using a 4-parameter Hill model via the QPI microscope system 110 for all conditions are summarized in Tables 5-8 below. To determine if there is a response, as illustrated by FIG. 2B, the QPI microscope system 110 tests goodness of fit, adjusts for degrees of freedom using an F-test at significance p<0.01 by comparison to a flat line, as an indicator of no response. For cases with a better fit to the Hill equation, the 4-parameter Hill curve may be used by the QPI microscope system 110 to determine the EC₅₀, a measure of sensitivity to therapy because it represents the effective concentration at which 50% of the cells respond to therapy, as illustrated by FIG. 10. FIG. 10 is shows graphs 2000 which include dose response plots that show mean specific growth rate as a function of concentration. A 4-parameter Hill curve is fit to each dose response plot to extract the depth of response and EC₅₀ for each cell/drug pair. A flat line represents no response due to failure of the F-test to reject the null hypothesis that the logistic fit is indistinguishable from a flat line. For example, FIG. 2B is graph 1205 including a dose response curve representing specific growth rate of cells of 450-700 MCF-7 cell clusters averaged over 27 individual imaging locations used to fit a 4-point Hill equation to dose response data for measurement of EC₅₀ and depth of response. Each color indicates a different drug or therapy used. Cytostatic response is noted as a moderate decrease in SGR, while cytostatic responses are indicated by a minimum asymptote of the hill curve at or below zero. The EC₅₀ for a particular therapy is the inflection point of the Hill curve, and one of the fitting parameters in the Hill equation.

TABLE 5
Summary of 72 h QPI Fit Parameters.

| | | Max | Min | | Hill |
|---|---|---|---|---|---|
| Cell Line | Treatment | Asymptote | Asymptote | log₁₀(EC₅₀) | Slope |
| BT-474 | 4HT | −0.012243 | 0.0090524 | −1.2566 | 3.3746 |
| BT-474 | 4HT | −0.012123 | 0.007511 | −1.2595 | 3.7567 |
| BT-474 | 4HT | −0.011659 | 0.0080994 | −1.2511 | 4.6177 |
| BT-474 | Lapatinib | −0.0027173 | 0.0096462 | 0.05298 | 1.0735 |
| BT-474 | Lapatinib | −0.0052326 | 0.0086812 | −0.41887 | 0.8265 |
| BT-474 | Lapatinib | −0.0029677 | 0.0095191 | −0.07466 | 1.1159 |
| BT-474 | Fulvestrant | 0.0061526 | 0.0098544 | −0.55128 | 1.943 |
| BT-474 | Fulvestrant | 0.0021989 | 0.0084919 | −1.2451 | 0.90323 |
| BT-474 | Fulvestrant | 0.003561 | 0.009456 | −1.1396 | 1.181 |
| BT-474 | Doxorubicin | −0.0035904 | 0.0096261 | 0.32357 | 3.7081 |
| BT-474 | Doxorubicin | −0.0038072 | 0.0091753 | 0.33612 | 4.795 |
| BT-474 | Doxorubicin | −0.0036956 | 0.0093179 | 0.31864 | 3.6266 |
| BT-474 | 5FU | NaN | NaN | NaN | NaN |
| BT-474 | 5FU | 0.0077083 | 0.0086434 | 1.2757 | 1.5745 |
| BT-474 | 5FU | 0.0077335 | 0.0096646 | 0.86675 | 1.1389 |
| BT-474 | Palbociclib | −0.012284 | 0.0088873 | −1.0904 | 1.7426 |
| BT-474 | Palbociclib | −0.021596 | 0.0088382 | −1.2071 | 1.3461 |
| BT-474 | Palbociclib | −0.01047 | 0.006465 | −1.2126 | 3.0929 |
| BT-474 | Vinblastine | −0.0016776 | 0.002895 | −1.2124 | 2.4178 |
| BT-474 | Vinblastine | −0.0022478 | 0.0035382 | −0.86494 | 1.3815 |
| BT-474 | Vinblastine | NaN | NaN | NaN | NaN |
| BT-474 | Carboplatin | NaN | NaN | NaN | NaN |
| BT-474 | Carboplatin | NaN | NaN | NaN | NaN |
| BT-474 | Carboplatin | NaN | NaN | NaN | NaN |
| BT-474 | Docetaxel | NaN | NaN | NaN | NaN |
| BT-474 | Docetaxel | 0.0016398 | 0.0093873 | 2.4173 | 3.8183 |
| BT-474 | Docetaxel | 0.0027019 | 0.0074641 | 2.1891 | 2.5139 |
| MCF-7 | 4HT | −0.036204 | 0.021371 | −2.2979 | 0.49996 |
| MCF-7 | 4HT | −0.030002 | 0.020942 | −1.3073 | 3.4317 |
| MCF-7 | 4HT | −0.0017971 | 0.024985 | −0.83625 | 2.0283 |
| MCF-7 | Lapatinib | NaN | NaN | NaN | NaN |
| MCF-7 | Lapatinib | 0.016595 | 0.02661 | −0.25607 | 1.4826 |
| MCF-7 | Lapatinib | NaN | NaN | NaN | NaN |
| MCF-7 | Fulvestrant | 0.016563 | 0.023766 | 0.46959 | 0.55623 |
| MCF-7 | Fulvestrant | 0.015733 | 0.028109 | 1.6624 | 0.74493 |
| MCF-7 | Fulvestrant | NaN | NaN | NaN | NaN |
| MCF-7 | Doxorubicin | −0.0044947 | 0.024871 | 0.62871 | 1.1171 |
| MCF-7 | Doxorubicin | −0.0016633 | 0.026965 | 0.71971 | 1.2782 |
| MCF-7 | Doxorubicin | −0.0010716 | 0.02807 | 0.70115 | 1.7872 |
| MCF-7 | 5FU | 0.0077456 | 0.023197 | −1.0204 | 1.9144 |
| MCF-7 | 5FU | −0.013111 | 0.02426 | −1.7232 | 0.50302 |
| MCF-7 | 5FU | 0.0045658 | 0.028664 | −1.1392 | 2.7373 |
| MCF-7 | Palbociclib | 0.0094794 | 0.017756 | 0.23914 | 1.1602 |
| MCF-7 | Palbociclib | 0.012917 | 0.021279 | 0.91375 | 4.6201 |
| MCF-7 | Palbociclib | NaN | NaN | NaN | NaN |
| MCF-7 | Vinblastine | −0.0029768 | 0.019792 | 2.6102 | 0.3759 |
| MCF-7 | Vinblastine | 0.00044082 | 0.028416 | 3.1775 | 0.38079 |
| MCF-7 | Vinblastine | −7.10E−05 | 0.02817 | 3.3758 | 0.43076 |
| MCF-7 | Carboplatin | 0.0070474 | 0.019941 | −1.2925 | 3.7715 |
| MCF-7 | Carboplatin | 0.011416 | 0.02292 | −0.94306 | 2.34 |
| MCF-7 | Carboplatin | NaN | NaN | NaN | NaN |
| MCF-7 | Docetaxel | 0.0040802 | 0.020998 | 3.173 | 1.6038 |
| MCF-7 | Docetaxel | 0.0058158 | 0.025674 | 3.0868 | 0.92483 |
| MCF-7 | Docetaxel | 0.0044879 | 0.018804 | 2.8709 | 1.3228 |
| MDA-MB- | 4HT | −0.0061344 | 0.02885 | −1.2344 | 2.0951 |
| 231 | | | | | |
| MDA-MB- | 4HT | −0.015061 | 0.030289 | −1.2249 | 2.4347 |
| 231 | | | | | |
| MDA-MB- | 4HT | −0.0084546 | 0.032461 | −1.0505 | 1.6101 |
| 231 | | | | | |
| MDA-MB- | Lapatinib | NaN | NaN | NaN | NaN |
| 231 | | | | | |
| MDA-MB- | Lapatinib | NaN | NaN | NaN | NaN |
| 231 | | | | | |
| MDA-MB- | Lapatinib | NaN | NaN | NaN | NaN |
| 231 | | | | | |
| MDA-MB- | Fulvestrant | 0.014234 | 0.027957 | −0.83887 | 1.492 |
| 231 | | | | | |
| MDA-MB- | Fulvestrant | 0.0090702 | 0.030323 | −1.2025 | 0.9134 |
| 231 | | | | | |
| MDA-MB- | Fulvestrant | NaN | NaN | NaN | NaN |
| 231 | | | | | |
| MDA-MB- | Doxorubicin | 0.00023227 | 0.032911 | 1.1002 | 0.99821 |
| 231 | | | | | |
| MDA-MB- | Doxorubicin | 0.0021831 | 0.031359 | 1.0414 | 1.1886 |
| 231 | | | | | |
| MDA-MB- | Doxorubicin | NaN | NaN | NaN | NaN |
| 231 | | | | | |
| MDA-MB- | 5FU | NaN | NaN | NaN | NaN |
| 231 | | | | | |
| MDA-MB- | 5FU | NaN | NaN | NaN | NaN |
| 231 | | | | | |
| MDA-MB- | 5FU | −0.0096665 | 0.033508 | −2.0719 | 0.44826 |
| 231 | | | | | |
| MDA-MB- | Palbociclib | −2.92E−05 | 0.034691 | 0.53285 | 0.39043 |
| 231 | | | | | |
| MDA-MB- | Palbociclib | 0.006396 | 0.03153 | 0.39856 | 0.56746 |
| 231 | | | | | |
| MDA-MB- | Palbociclib | 0.0077838 | 0.026635 | 0.18998 | 0.80828 |
| 231 | | | | | |
| MDA-MB- | Vinblastine | 0.010502 | 0.018709 | 1.5722 | 3.181 |
| 231 | | | | | |
| MDA-MB- | Vinblastine | 0.0079383 | 0.030861 | 2.0426 | 1.2208 |
| 231 | | | | | |
| MDA-MB- | Vinblastine | 0.0076537 | 0.030013 | 2.4016 | 1.0819 |
| 231 | | | | | |
| MDA-MB- | Carboplatin | 0.016091 | 0.028606 | −0.97831 | 1.1951 |
| 231 | | | | | |
| MDA-MB- | Carboplatin | 0.023034 | 0.030446 | −0.20744 | 1.0461 |
| 231 | | | | | |
| MDA-MB- | Carboplatin | 0.020918 | 0.02714 | −1.2064 | 3.6722 |
| 231 | | | | | |
| MDA-MB- | Docetaxel | 0.013818 | 0.040276 | 3.7796 | 0.68569 |
| 231 | | | | | |
| MDA-MB- | Docetaxel | 0.010167 | 0.029619 | 2.6543 | 1.2756 |
| 231 | | | | | |
| MDA-MB- | Docetaxel | 0.027423 | 0.027423 | 0.027423 | 0.027423 |
| 231 | | | | | |

TABLE 6
Summary of 72 h CTG Fit Parameters.

| | | Min | | |
|---|---|---|---|---|
| Cell Line | Treatment | Asymptote | log₁₀(EC₅₀) | Hill Slope |
| BT-474 | 4HT | 1.76E−08 | −0.6116009 | 3.31212079 |
| BT-474 | 4HT | 8.28E−08 | −0.6471363 | 3.65542399 |
| BT-474 | 4HT | 1.76E−08 | −0.6116009 | 3.31212079 |
| BT-474 | Lapatinib | 0.40619444 | 0.04317705 | 1.02078897 |
| BT-474 | Lapatinib | 0.31019863 | −0.4776065 | 0.67355424 |
| BT-474 | Lapatinib | 0.40619444 | 0.04317705 | 1.02078897 |
| BT-474 | Fulvestrant | 0.16084965 | −3.3007158 | 0.4429625 |
| BT-474 | Fulvestrant | 0.80089054 | −1.4766613 | 0.16076134 |
| BT-474 | Fulvestrant | 0.16084965 | −3.3007158 | 0.4429625 |
| BT-474 | Doxorubicin | 0.54070803 | 0.36288053 | 8.99890339 |
| BT-474 | Doxorubicin | 0.56016172 | 0.34554189 | 8.65192829 |
| BT-474 | Doxorubicin | 0.54070803 | 0.36288053 | 8.99890339 |
| BT-474 | 5FU | 0.9379763 | 0.76668875 | 1.18468212 |
| BT-474 | 5FU | 0.92220471 | 1.89894954 | 1.04196332 |
| BT-474 | 5FU | 0.9379763 | 0.76668875 | 1.18468212 |
| BT-474 | Palbociclib | 0.0162794 | −0.6074917 | 5.46658959 |
| BT-474 | Palbociclib | 0.01895142 | −0.9431269 | 7.80155043 |
| BT-474 | Palbociclib | | | |
| BT-474 | Vinblastine | NaN | NaN | NaN |
| BT-474 | Vinblastine | 0.06356225 | 2.11588039 | 0.1 |
| BT-474 | Vinblastine | | | |
| BT-474 | Carboplatin | NaN | NaN | NaN |
| BT-474 | Carboplatin | NaN | NaN | NaN |
| BT-474 | Carboplatin | | | |
| BT-474 | Docetaxel | 0.28048817 | 2.47599228 | 7.93795064 |
| BT-474 | Docetaxel | 0.33482815 | 2.40279016 | 5.66701432 |
| BT-474 | Docetaxel | | | |
| MCF-7 | 4HT | 2.23E−14 | −0.5373351 | 1.22252746 |
| MCF-7 | 4HT | 2.22E−14 | −0.5548992 | 1.70248307 |
| MCF-7 | 4HT | 3.01E−12 | −0.6776176 | 0.55376953 |
| MCF-7 | Lapatinib | NaN | NaN | NaN |
| MCF-7 | Lapatinib | NaN | NaN | NaN |
| MCF-7 | Lapatinib | NaN | NaN | NaN |
| MCF-7 | Fulvestrant | 0.75997456 | 1.11171565 | 3.23989902 |
| MCF-7 | Fulvestrant | 0.70729731 | 0.54750828 | 0.61996047 |
| MCF-7 | Fulvestrant | 0.69560712 | −0.0290374 | 1.13470018 |
| MCF-7 | Doxorubicin | 0.05061197 | 0.77668887 | 1.33341023 |
| MCF-7 | Doxorubicin | 0.06352832 | 0.82191032 | 1.2404152 |
| MCF-7 | Doxorubicin | 0.02696597 | 0.73664212 | 1.13003334 |
| MCF-7 | 5FU | 0.19521382 | −0.8373439 | 1.25433119 |
| MCF-7 | 5FU | 0.00365251 | −1.0391871 | 1.05946801 |
| MCF-7 | 5FU | 0.01677516 | −1.1628017 | 1.20243058 |
| MCF-7 | Palbociclib | 0.12437857 | −0.2098015 | 0.76167769 |
| MCF-7 | Palbociclib | 0.22100217 | −0.1875723 | 0.90042557 |
| MCF-7 | Palbociclib | 0.12437857 | −0.2098015 | 0.76167769 |
| MCF-7 | Vinblastine | 4.32E−11 | 2.66825461 | 0.25723914 |
| MCF-7 | Vinblastine | 1.18E−11 | 2.68860135 | 0.22174203 |
| MCF-7 | Vinblastine | 4.32E−11 | 2.66825461 | 0.25723914 |
| MCF-7 | Carboplatin | 0.50475108 | −1.2008433 | 3.1255046 |
| MCF-7 | Carboplatin | 0.64977548 | −1.1719818 | 5.11412756 |
| MCF-7 | Carboplatin | 0.50475108 | −1.2008433 | 3.1255046 |
| MCF-7 | Docetaxel | 0.36798277 | 3.34203858 | 5.07903298 |
| MCF-7 | Docetaxel | 0.40645514 | 3.2910087 | 4.96531284 |
| MCF-7 | Docetaxel | 0.36798277 | 3.34203858 | 5.07903298 |
| MDA-MB- | 4HT | 0.00331525 | −0.4994514 | 6.83002316 |
| 231 | | | | |
| MDA-MB- | 4HT | 0.01516022 | −0.9458016 | 2.24713174 |
| 231 | | | | |
| MDA-MB- | 4HT | 0.01516022 | −0.9458016 | 2.24713174 |
| 231 | | | | |
| MDA-MB- | Lapatinib | NaN | NaN | NaN |
| 231 | | | | |
| MDA-MB- | Lapatinib | NaN | NaN | NaN |
| 231 | | | | |
| MDA-MB- | Lapatinib | NaN | NaN | NaN |
| 231 | | | | |
| MDA-MB- | Fulvestrant | NaN | NaN | NaN |
| 231 | | | | |
| MDA-MB- | Fulvestrant | 0.01999134 | −1.4975842 | 0.76411654 |
| 231 | | | | |
| MDA-MB- | Fulvestrant | 0.01999134 | −1.4975842 | 0.76411654 |
| 231 | | | | |
| MDA-MB- | Doxorubicin | 0.01710846 | 0.69023834 | 0.8063624 |
| 231 | | | | |
| MDA-MB- | Doxorubicin | 0.03777255 | 0.54005797 | 0.79057635 |
| 231 | | | | |
| MDA-MB- | Doxorubicin | 0.03777255 | 0.54005797 | 0.79057635 |
| 231 | | | | |
| MDA-MB- | 5FU | 0.49374026 | −0.7780416 | 1.99739946 |
| 231 | | | | |
| MDA-MB- | 5FU | NaN | NaN | NaN |
| 231 | | | | |
| MDA-MB- | 5FU | NaN | NaN | NaN |
| 231 | | | | |
| MDA-MB- | Palbociclib | 0.01895142 | −0.9431269 | 7.80155043 |
| 231 | | | | |
| MDA-MB- | Palbociclib | 7.98E−09 | −0.5659168 | 0.89487981 |
| 231 | | | | |
| MDA-MB- | Palbociclib | | | |
| 231 | | | | |
| MDA-MB- | Vinblastine | 0.06356225 | 2.11588039 | 0.1 |
| 231 | | | | |
| MDA-MB- | Vinblastine | 0.29107249 | 2.45867246 | 1.59255449 |
| 231 | | | | |
| MDA-MB- | Vinblastine | | | |
| 231 | | | | |
| MDA-MB- | Carboplatin | NaN | NaN | NaN |
| 231 | | | | |
| MDA-MB- | Carboplatin | NaN | NaN | NaN |
| 231 | | | | |
| MDA-MB- | Carboplatin | | | |
| 231 | | | | |
| MDA-MB- | Docetaxel | 0.33482815 | 2.40279016 | 5.66701432 |
| 231 | | | | |
| MDA-MB- | Docetaxel | 0.25612136 | 2.34227579 | 1.02215601 |
| 231 | | | | |
| MDA-MB- | Docetaxel | | | |
| 231 | | | | |

TABLE 7
Summary of 24 h QPI Fit Parameters.

| Cell Line | Treatment | Max Asymptote | Min Asymptote | log₁₀(EC₅₀) | Hill Slope |
|---|---|---|---|---|---|
| BT-474 | 4HT | 0.012322 | −0.016126 | −1.1593 | 2.303 |
| BT-474 | 4HT | 0.0096903 | −0.023754 | −1.2498 | 3.2307 |
| BT-474 | 4HT | 0.009107 | −0.024931 | −1.2667 | 3.3723 |
| BT-474 | Lapatinib | 0.012392 | −0.0017672 | 0.041503 | 1.0622 |
| BT-474 | Lapatinib | 0.010121 | −0.0065696 | −0.45592 | 0.76828 |
| BT-474 | Lapatinib | 0.0091144 | −0.004991 | −0.18635 | 1.0728 |
| BT-474 | Fulvestrant | NaN | NaN | NaN | NaN |
| BT-474 | Fulvestrant | NaN | NaN | NaN | NaN |
| BT-474 | Fulvestrant | 0.010593 | 0.0073826 | −1.1373 | 0.54479 |
| BT-474 | Doxorubicin | 0.012969 | −0.0015671 | −0.25116 | 1.5047 |
| BT-474 | Doxorubicin | 0.010507 | −0.0025059 | −0.28271 | 1.6529 |
| BT-474 | Doxorubicin | 0.00993 | −0.0023714 | −0.22345 | 1.3096 |
| BT-474 | 5FU | NaN | NaN | NaN | NaN |
| BT-474 | 5FU | NaN | NaN | NaN | NaN |
| BT-474 | 5FU | 0.011075 | 0.0090206 | 1.1661 | 0.91345 |
| BT-474 | Palbociclib | 0.010824 | −0.017618 | −1.1436 | 2.2846 |
| BT-474 | Palbociclib | 0.0085557 | −0.027698 | −1.2331 | 1.7745 |
| BT-474 | Palbociclib | 0.0088152 | −0.013936 | −1.2554 | 2.7163 |
| BT-474 | Vinblastine | NaN | NaN | NaN | NaN |
| BT-474 | Vinblastine | 0.0075818 | −0.0048122 | −1.6886 | 0.40517 |
| BT-474 | Vinblastine | NaN | NaN | NaN | NaN |
| BT-474 | Carboplatin | NaN | NaN | NaN | NaN |
| BT-474 | Carboplatin | NaN | NaN | NaN | NaN |
| BT-474 | Carboplatin | NaN | NaN | NaN | NaN |
| BT-474 | Docetaxel | NaN | NaN | NaN | NaN |
| BT-474 | Docetaxel | 0.0098739 | 0.0051801 | 0.954 | 0.2745 |
| BT-474 | Docetaxel | 0.01051 | 0.0080302 | 0.4202 | 0.53609 |
| MCF-7 | 4HT | 0.027529 | −0.013962 | −2.5084 | 0.28742 |
| MCF-7 | 4HT | 0.025051 | −0.048137 | −1.3545 | 4.2771 |
| MCF-7 | 4HT | 0.025051 | −0.048137 | −1.3545 | 4.2771 |
| MCF-7 | Lapatinib | NaN | NaN | NaN | NaN |
| MCF-7 | Lapatinib | 0.029189 | 0.020597 | −0.28254 | 1.4871 |
| MCF-7 | Lapatinib | NaN | NaN | NaN | NaN |
| MCF-7 | Fulvestrant | NaN | NaN | NaN | NaN |
| MCF-7 | Fulvestrant | 0.041687 | 0.020209 | 3.0581 | 0.45366 |
| MCF-7 | Fulvestrant | NaN | NaN | NaN | NaN |
| MCF-7 | Doxorubicin | 0.030238 | −0.023067 | −0.79251 | 0.42248 |
| MCF-7 | Doxorubicin | 0.036141 | −0.0037048 | 0.25807 | 0.50563 |
| MCF-7 | Doxorubicin | 0.036141 | −0.0037048 | 0.25807 | 0.50563 |
| MCF-7 | 5FU | 0.026759 | 0.013818 | −0.933 | 1.4172 |
| MCF-7 | 5FU | 0.026648 | 0.0074358 | −1.151 | 0.61703 |
| MCF-7 | 5FU | 0.026648 | 0.0074358 | −1.151 | 0.61703 |
| MCF-7 | Palbociclib | 0.021687 | 0.016566 | 0.64443 | 1.6152 |
| MCF-7 | Palbociclib | 0.030539 | 0.020431 | 1.6083 | 1.0687 |
| MCF-7 | Palbociclib | NaN | NaN | NaN | NaN |
| MCF-7 | Vinblastine | NaN | NaN | NaN | NaN |
| MCF-7 | Vinblastine | NaN | NaN | NaN | NaN |
| MCF-7 | Vinblastine | 0.019577 | 0.0057957 | 2.8137 | 3.2016 |
| MCF-7 | Carboplatin | NaN | NaN | NaN | NaN |
| MCF-7 | Carboplatin | NaN | NaN | NaN | NaN |
| MCF-7 | Carboplatin | NaN | NaN | NaN | NaN |
| MCF-7 | Docetaxel | 0.024571 | 0.010568 | 2.8284 | 1.6196 |
| MCF-7 | Docetaxel | 0.028914 | 0.01168 | 2.493 | 1.4393 |
| MCF-7 | Docetaxel | 0.022135 | 0.0081436 | 2.7801 | 1.4829 |
| MDA-MB- | 4HT | 0.028747 | 0.0076759 | −1.2332 | 2.9327 |
| 231 | | | | | |
| MDA-MB- | 4HT | 0.031066 | 0.0076109 | −1.2248 | 3.1352 |
| 231 | | | | | |
| MDA-MB- | 4HT | 0.033032 | −0.0030236 | −1.2436 | 1.11 |
| 231 | | | | | |
| MDA-MB- | Lapatinib | NaN | NaN | NaN | NaN |
| 231 | | | | | |
| MDA-MB- | Lapatinib | NaN | NaN | NaN | NaN |
| 231 | | | | | |
| MDA-MB- | Lapatinib | NaN | NaN | NaN | NaN |
| 231 | | | | | |
| MDA-MB- | Fulvestrant | 0.028472 | 0.024009 | −0.5352 | 2.2482 |
| 231 | | | | | |
| MDA-MB- | Fulvestrant | NaN | NaN | NaN | NaN |
| 231 | | | | | |
| MDA-MB- | Fulvestrant | NaN | NaN | NaN | NaN |
| 231 | | | | | |
| MDA-MB- | Doxorubicin | 0.031227 | 0.0022289 | 0.51826 | 1.1528 |
| 231 | | | | | |
| MDA-MB- | Doxorubicin | 0.034917 | 0.0064762 | 0.83658 | 1.2328 |
| 231 | | | | | |
| MDA-MB- | Doxorubicin | 0.034443 | 0.0034762 | 0.67043 | 1.1155 |
| 231 | | | | | |
| MDA-MB- | 5FU | NaN | NaN | NaN | NaN |
| 231 | | | | | |
| MDA-MB- | 5FU | NaN | NaN | NaN | NaN |
| 231 | | | | | |
| MDA-MB- | 5FU | 0.033362 | 0.01996 | −1.2414 | 0.34096 |
| 231 | | | | | |
| MDA-MB- | Palbociclib | 0.034586 | 0.0047811 | 0.11465 | 0.27023 |
| 231 | | | | | |
| MDA-MB- | Palbociclib | 0.033015 | −0.016223 | −2.5174 | 0.23714 |
| 231 | | | | | |
| MDA-MB- | Palbociclib | 0.023222 | 0.011659 | −0.84161 | 1.0503 |
| 231 | | | | | |
| MDA-MB- | Vinblastine | 0.024194 | 0.014904 | 1.7015 | 3.4836 |
| 231 | | | | | |
| MDA-MB- | Vinblastine | 0.028244 | 0.015028 | 1.9535 | 2.9411 |
| 231 | | | | | |
| MDA-MB- | Vinblastine | 0.031241 | 0.015169 | 2.8902 | 0.88238 |
| 231 | | | | | |
| MDA-MB- | Carboplatin | NaN | NaN | NaN | NaN |
| 231 | | | | | |
| MDA-MB- | Carboplatin | NaN | NaN | NaN | NaN |
| 231 | | | | | |
| MDA-MB- | Carboplatin | NaN | NaN | NaN | NaN |
| 231 | | | | | |
| MDA-MB- | Docetaxel | 0.033465 | 0.016726 | 2.5068 | 0.8657 |
| 231 | | | | | |
| MDA-MB- | Docetaxel | 0.030658 | 0.015784 | 1.9777 | 1.9958 |
| 231 | | | | | |
| MDA-MB- | Docetaxel | 0.023636 | 0.023636 | 0.023636 | 0.023636 |
| 231 | | | | | |

TABLE 8
Summary of 24 h CTG Fit Parameters.

| Cell Line | Treatment | Min Asymptote | log₁₀(EC₅₀) | Hill Slope |
|---|---|---|---|---|
| BT-474 | 4HT | 0.079420198 | −0.736012 | 4.59596307 |
| BT-474 | 4HT | 0.079420198 | −0.736012 | 4.59596307 |
| BT-474 | 4HT | | | |
| BT-474 | Lapatinib | 0.292598089 | −2.2177382 | 0.45754316 |
| BT-474 | Lapatinib | 0.292598089 | −2.2177382 | 0.45754316 |
| BT-474 | Lapatinib | | | |
| BT-474 | Fulvestrant | NaN | NaN | NaN |
| BT-474 | Fulvestrant | NaN | NaN | NaN |
| BT-474 | Fulvestrant | | | |
| BT-474 | Doxorubicin | NaN | NaN | NaN |
| BT-474 | Doxorubicin | NaN | NaN | NaN |
| BT-474 | Doxorubicin | | | |
| BT-474 | 5FU | 0.925043471 | −1.0374941 | 0.53878348 |
| BT-474 | 5FU | 0.925043471 | −1.0374941 | 0.53878348 |
| BT-474 | 5FU | | | |
| BT-474 | Palbociclib | 0.220178289 | −1.1514272 | 6.94504091 |
| BT-474 | Palbociclib | | | |
| BT-474 | Palbociclib | | | |
| BT-474 | Vinblastine | 0.565868697 | −0.2618522 | 0.34452378 |
| BT-474 | Vinblastine | | | |
| BT-474 | Vinblastine | | | |
| BT-474 | Carboplatin | NaN | NaN | NaN |
| BT-474 | Carboplatin | | | |
| BT-474 | Carboplatin | | | |
| BT-474 | Docetaxel | 0.84906734 | 1.86413446 | 7.17467304 |
| BT-474 | Docetaxel | | | |
| BT-474 | Docetaxel | | | |
| MCF-7 | 4HT | 7.96E−12 | −1.009347 | 1.32007534 |
| MCF-7 | 4HT | 5.22E−09 | −1.5155591 | 0.56737708 |
| MCF-7 | 4HT | | | |
| MCF-7 | Lapatinib | NaN | NaN | NaN |
| MCF-7 | Lapatinib | NaN | NaN | NaN |
| MCF-7 | Lapatinib | | | |
| MCF-7 | Fulvestrant | 0.518906922 | −3.3010256 | 0.31765435 |
| MCF-7 | Fulvestrant | 0.944643498 | 0.37760685 | 8.19369233 |
| MCF-7 | Fulvestrant | | | |
| MCF-7 | Doxorubicin | 0.560631684 | −0.2880774 | 7.54355058 |
| MCF-7 | Doxorubicin | 0.276796413 | −0.3063815 | 1.39021658 |
| MCF-7 | Doxorubicin | | | |
| MCF-7 | 5FU | NaN | NaN | NaN |
| MCF-7 | 5FU | 0.923793359 | −0.2769199 | 7.0805257 |
| MCF-7 | 5FU | | | |
| MCF-7 | Palbociclib | | | |
| MCF-7 | Palbociclib | | | |
| MCF-7 | Palbociclib | | | |
| MCF-7 | Vinblastine | | | |
| MCF-7 | Vinblastine | | | |
| MCF-7 | Vinblastine | | | |
| MCF-7 | Carboplatin | | | |
| MCF-7 | Carboplatin | | | |
| MCF-7 | Carboplatin | | | |
| MCF-7 | Docetaxel | | | |
| MCF-7 | Docetaxel | | | |
| MCF-7 | Docetaxel | | | |
| MDA-MB- | 4HT | 0.392990605 | −0.5157713 | 5.35070907 |
| 231 | | | | |
| MDA-MB- | 4HT | | | |
| 231 | | | | |
| MDA-MB- | 4HT | | | |
| 231 | | | | |
| MDA-MB- | Lapatinib | NaN | NaN | NaN |
| 231 | | | | |
| MDA-MB- | Lapatinib | | | |
| 231 | | | | |
| MDA-MB- | Lapatinib | | | |
| 231 | | | | |
| MDA-MB- | Fulvestrant | NaN | NaN | NaN |
| 231 | | | | |
| MDA-MB- | Fulvestrant | | | |
| 231 | | | | |
| MDA-MB- | Fulvestrant | | | |
| 231 | | | | |
| MDA-MB- | Doxorubicin | 0.407151354 | −0.0954985 | 1.12524079 |
| 231 | | | | |
| MDA-MB- | Doxorubicin | | | |
| 231 | | | | |
| MDA-MB- | Doxorubicin | | | |
| 231 | | | | |
| MDA-MB- | 5FU | NaN | NaN | NaN |
| 231 | | | | |
| MDA-MB- | 5FU | | | |
| 231 | | | | |
| MDA-MB- | 5FU | | | |
| 231 | | | | |
| MDA-MB- | Palbociclib | 0.220178289 | −1.1514272 | 6.94504091 |
| 231 | | | | |
| MDA-MB- | Palbociclib | | | |
| 231 | | | | |
| MDA-MB- | Palbociclib | | | |
| 231 | | | | |
| MDA-MB- | Vinblastine | 0.565868697 | −0.2618522 | 0.34452378 |
| 231 | | | | |
| MDA-MB- | Vinblastine | | | |
| 231 | | | | |
| MDA-MB- | Vinblastine | | | |
| 231 | | | | |
| MDA-MB- | Carboplatin | NaN | NaN | NaN |
| 231 | | | | |
| MDA-MB- | Carboplatin | | | |
| 231 | | | | |
| MDA-MB- | Carboplatin | | | |
| 231 | | | | |
| MDA-MB- | Docetaxel | 0.84906734 | 1.86413446 | 7.17467304 |
| 231 | | | | |
| MDA-MB- | Docetaxel | | | |
| 231 | | | | |
| MDA-MB- | Docetaxel | | | |
| 231 | | | | |

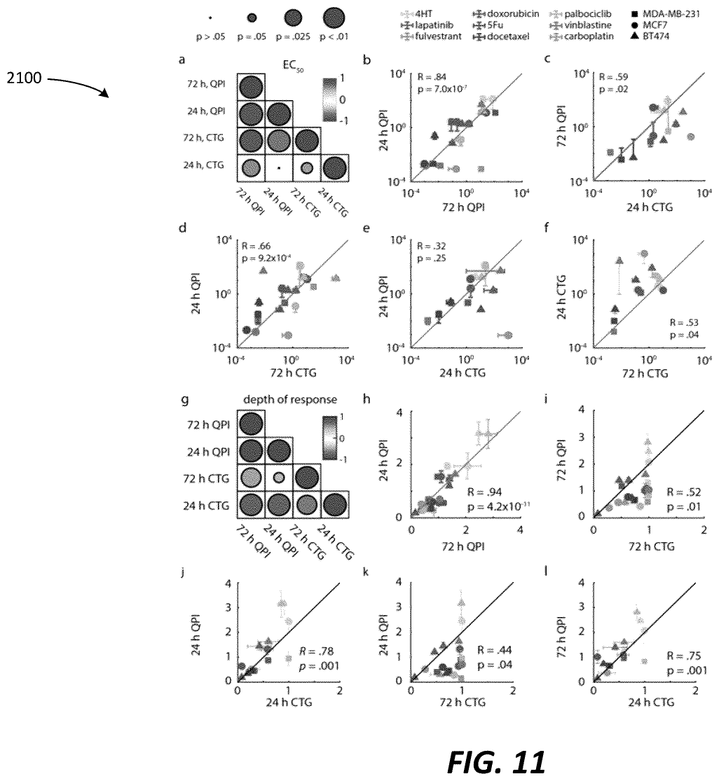

For conditions with a response, the QPI microscope system 110 determines DoR from the fitted Hill curve as the difference between the asymptotes at the highest and lowest concentrations, normalized by the asymptote at low concentration. The normalization accounts for differences in the control growth rates of each cell line. With reference to FIG. 2B, the QPI microscope system 110 uses the DoR to determine how toxic a therapy is to a particular cell line. Now referring to FIG. 2C, a DoR less than 1 represents a cytostatic response with reduced cell growth relative to control and a DoR greater than 1 represents a cytotoxic response resulting in loss of mass and is associated with cell death. For example, FIG. 2C is a graph 1210 of average cell mass normalized by initial cell mass of each cell cluster versus time for 486 clusters in response to 20 μM doxorubicin treatment, 615 clusters in response to 20 μM fulvestrant, and 476 clusters treated with DMSO control. In some instances, cytostatic response can be observed as the population slows cell mass accumulation (fulvestrant) while a cytotoxic response results in a gradual loss of cell mass due to cell death (doxorubicin). As illustrated by FIG. 2D, the EC₅₀ measured using the QPI microscope system 110 is highly concordant to the EC₅₀ measured using 72 h CTG as a gold standard of drug response with a correlation coefficient of 0.83 (p<0.001) and concordance coefficient of 0.84 (95% confidence interval=[0.57, 0.98]). For example, FIG. 2D is a graph 1215 of a comparison of EC₅₀ from a Hill equation fitting to CTG and QPI data from the QPI microscope system 110. The gray line shows an expected relationship (EC₅₀, CTG=EC₅₀,QPI). Each color indicates a different treatment therapy, and each symbol indicates a different cell line (Correlation coefficient, R=0.83, p=7.5×10-7, Concordance coefficient=0.84). Additionally, with reference to FIG. 2E, predictions of cell line/compound pairs that show no response (n=14) versus those showing a response (n=59) are highly concordant with CTG 72 h results (86%). When comparing QPI to CTG, concordance at 24 h and 72 h time points is generally high for EC₅₀ values but low for DoR, indicating that drugs can affect cell growth distinctly from measurable changes in ATP, as illustrated in FIG. 11. FIG. 11 shows graphs 2100 of 24 h QPI data predicted results in 72 h CTG and QPI experiments. Graph A of the graphs 2100 is a correlation map of EC₅₀ for both QPI and CTG experiments. Color indicates correlation (blue high, red low) and symbol size indicates p-value. Graph B of the graphs 2100 shows EC₅₀ values determined from 72 h QPI and from the first 24 h of QPI data are strongly correlated (R=0.84, p=7×10⁻⁷). Graph C of the graphs 2100 shows EC₅₀ values from 24 h QPI and 72 h CTG are strongly correlated (R=0.66, p=9.2×10⁻⁴). Graph D of the graphs 2100 shows EC₅₀ values measured in 72 h using QPI and 24 h CTG are moderately correlated (R=0.59, p=0.02). Graph E of the graphs 2100 shows ECso values obtained using 24 h of QPI data and 24 h CTG are not well correlated (R=0.32, p=0.25). Graph F of the graphs 2100 shows EC₅₀ values measured using 24 h CTG and 72 h CTG are moderately correlated (R=0.53, p=0.04). Graph G of the graphs 2100 is a correlation matrix of DoR measured using both QPI and CTG experiments. Color indicates correlation (blue high, red low) and symbol size indicates p-value. Graph H of the graphs 2100 shows DoR values measured in first 24 h of QPI and 72 h QPI are strongly correlated (R=0.71, p=1.4×10⁻⁴). Graph I of the graphs 2100 shows DoR determined using 72 h QPI and 24 h CTG are not well correlated (R=0.48, p=0.08). Graph J of the graphs 2100 shows the DoR values determined in the first 24 h of QPI and 24 h CTG are strongly correlated (R=0.71, p=3.0×10⁻³). Graph K of the graphs 2100 shows DoR measurements from the first 24 h of QPI and 72 h CTG are moderately correlated (R=0.5, p=0.02). Graph L of the graphs 2100 shows DoR measurements obtained using 72 h of QPI and 72 h CTG are moderately correlated (R=0.5, p=0.01). The gray line is y=x. The black line is y=2x. Error bars show SEM. For example, FIG. 2E is a confusion matrix 1220 of precision and accuracy of QPI relative to CTG. By comparing frequency, QPI predicts the same outcome as CTG. Error bars indicate standard error of the mean (SEM).

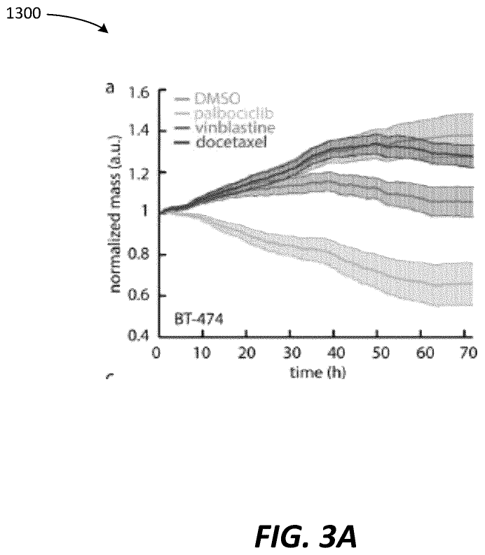

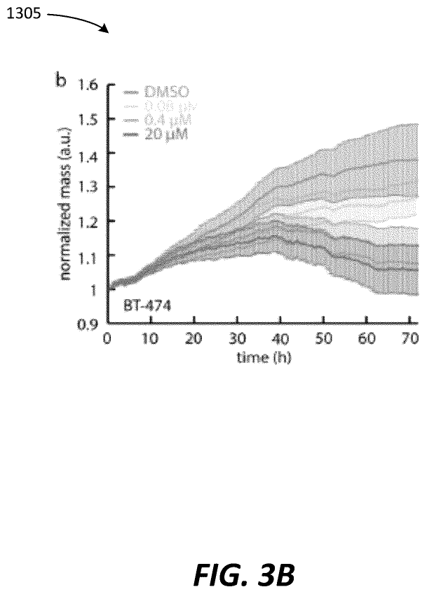

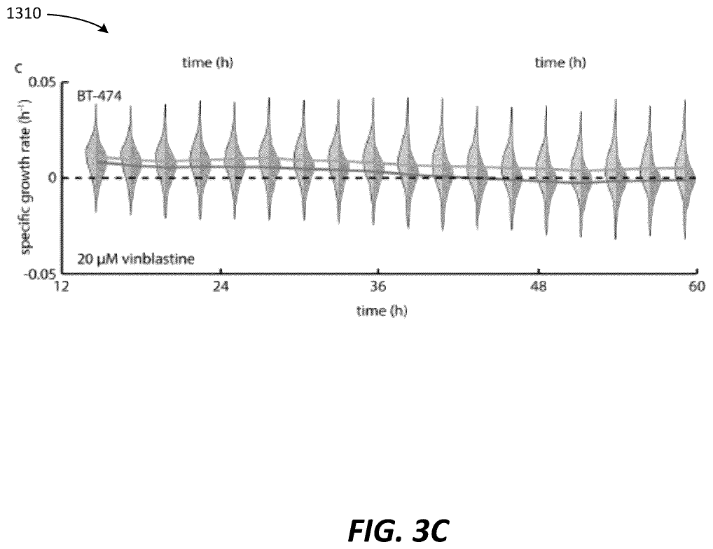

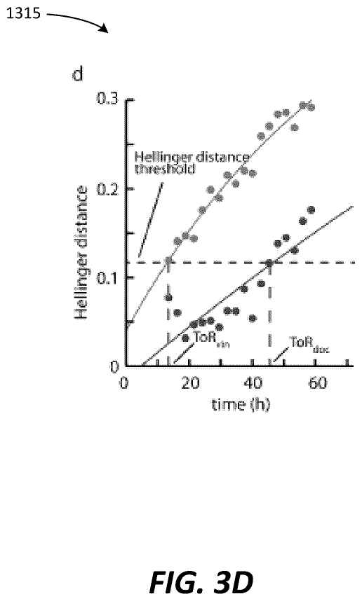

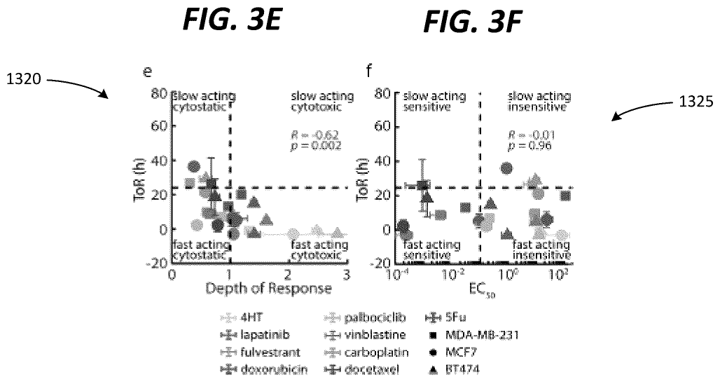

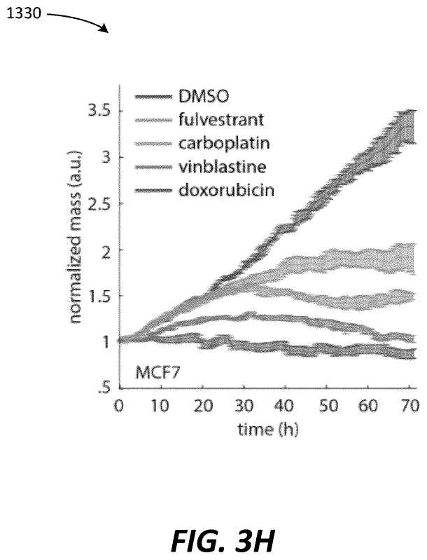

By precisely measuring the mass of individual cells during treatment, the QPI microscope system 110 gives rapid and sensitive insight into the dynamic response of cells to therapy throughout an experiment with high temporal resolution. FIG. 3A is a graph 1300 of mass versus time normalized by initial cell mass averaged over all BT-474 cell clusters at each time point, according to some embodiments. For example, as illustrated by the graph 1300 of FIG. 3A, the normalized mass versus time for BT-474 cells treated with 20 μM of palbociclib, 0.016 μM of docetaxel (nearest concentration to the measured EC₅₀), and 20 μM of vinblastine, the nearest concentration tested above the measured EC₅₀s, initially behave similarly to the control, but then exhibit differential, time-dependent responses. Of the noted conditions, palbociclib, a CDK 4/6 inhibitor that prevents the transition from G1 to S phase, elicits the fastest response, within 5 h from the start of imaging (8 h post-exposure), and with the largest DoR as indicated by a reduction in mass.

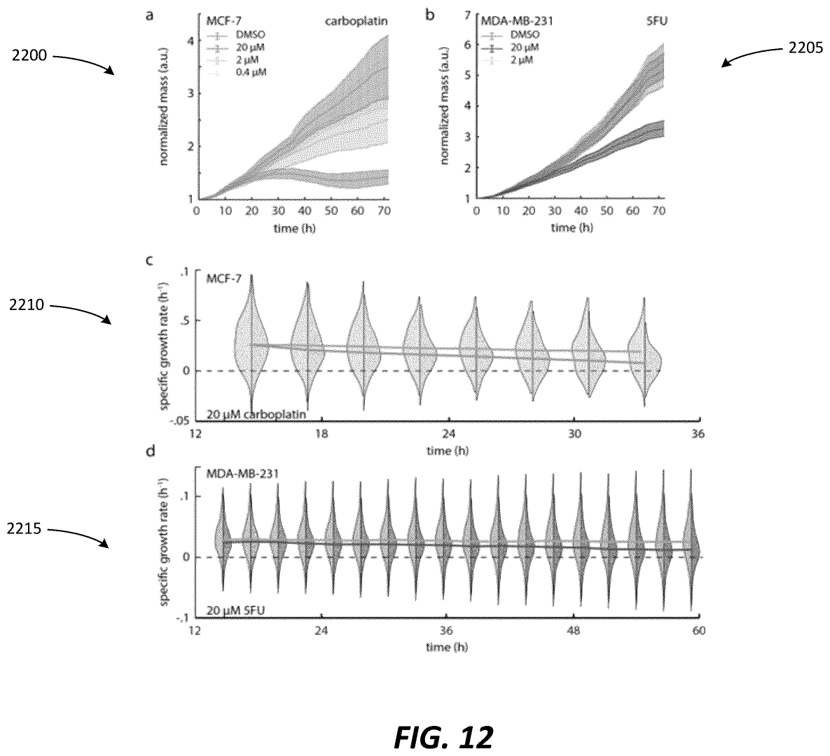

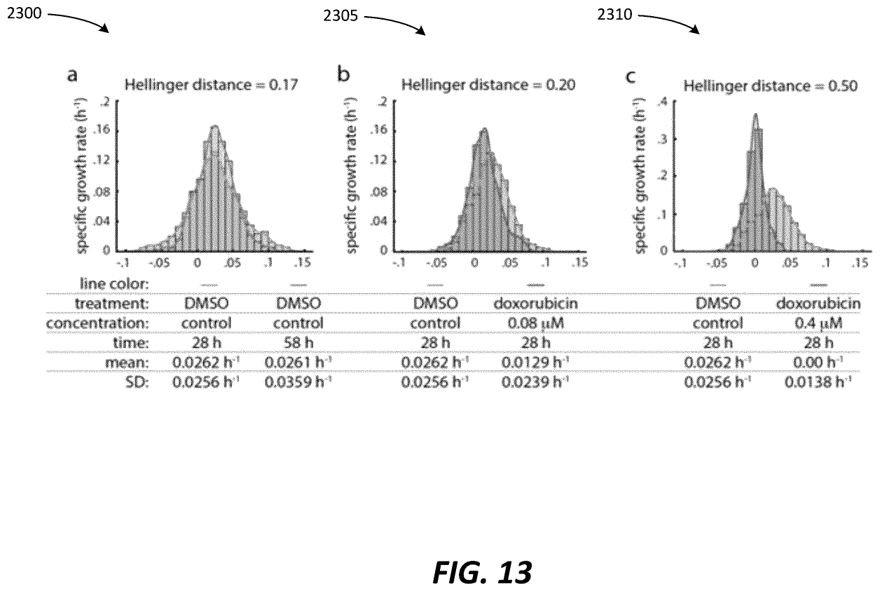

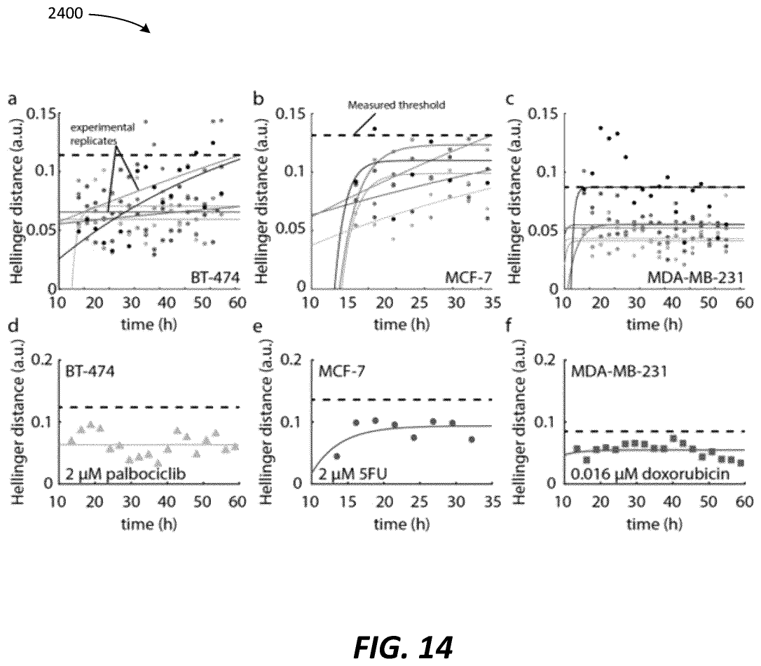

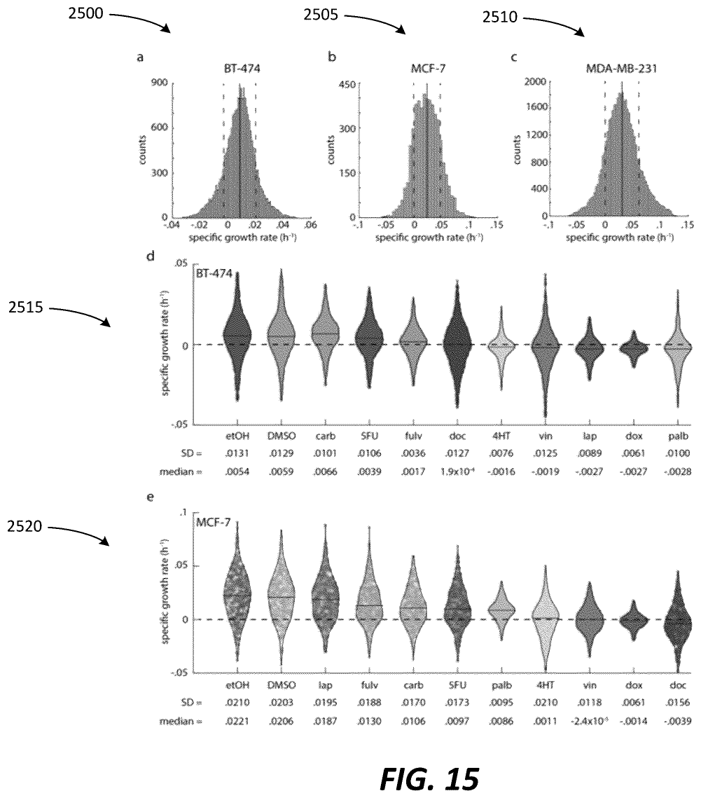

Referring now to FIG. 3B, temporal dynamics of response may also be dose dependent, with higher concentrations resulting in a substantially faster response than lower concentrations as larger proportion cells respond more quickly at higher doses, as shown by graphs 2200 and 2205 of FIG. 12. The graph 2200 of FIG. 12 shows average mass plotted against time, for carboplatin treated MCF-7 cells. Mass is normalized by the initial mass of each cell in the population. Dark orange represents 20 μM carboplatin treated cells, and lighter colors show the same plot for cells treated with lower concentrations. The graph 2205 of FIG. 12 shows average mass plotted against time for 5-fluorouracil (5FU) treated MDA-MB-231 cells. Mass is normalized by the initial mass of each cell in the population. Dark gray shows the normalized mass of cells treated with 20 μM 5FU and lighter gray shows cells treated with 2 μM 5FU. Error bars show SEM. For example, FIG. 3B is a graph 1305 of mass versus time normalized by the initial cell mass averaged over all cells at each time point for 0.08 μM, 0.4 μM, and 20 μM of vinblastine. To study response dynamics, changing distributions of SGR of single cells or cell clusters are examined as a function of time in FIG. 3C, as also shown by graphs 2210 and 2215 of FIG. 12. The graph 2210 of FIG. 12 shows MCF-7 response to 20 μM carboplatin (orange) versus DMSO control (blue) in 12 h bins showing an initially similar distribution that slowly begins to deviate over time. Solid lines represent the median of the distribution as a function of time. Dashed line shows SGR of zero. Individual data points shown within population distributions. The graph 2215 of FIG. 12 shows MDA-MB-231 response to 20 μM 5FU (gray) versus DMSO control (blue) in 12 h bins shows an initially similar distribution that slowly begins to deviate over time. Solid lines represent the median of the distribution as a function of time. Dashed line shows SGR of zero. Individual data points shown within population distributions. For example, FIG. 3C is a graph 1310 of BT-474 response. In the case of BT-474 cells treated with 20 μM vinblastine, for example, the average growth rate slowly decreases over 12-60 h of treatment, as indicated by a gradual separation of the control and treated cell populations in FIG. 3C. With continued reference to FIG. 3C, 20 μM vinblastine (magenta, n=1944 cells) versus DMSO control (blue, n=2414) in 24 h bins centered on different time points show an initially similar distribution that slowly begins to deviate over time. Solid lines represent the median of the distribution as a function of time. Individual data points show the specific growth rate of individual cells within population distributions. Referring now to FIG. 3D, a Hellinger distance may be used to quantify a time required for cells to respond to therapy. The Hellinger distance (e.g., a measure of the similarity between two probability distributions) provides a measure of the dynamic response of cells relative to any time-dependent changes in plate matched solvent controls and is impacted by changes in both the mean response as well as the variance of the distributions, as shown by graphs 2300-2310 of FIG. 13. Graph 2300 of FIG. 13 shows the specific growth rate distributions for DMSO treated MDA-MB-231 cells showing a small change during the experiment setting the threshold for DMSO treated cells in this experiment. The graph 2305 of FIG. 13 shows specific growth rate distribution of MDA-MB-231 cells treated with 0.08 μM of doxorubicin has a reduced mean and a Hellinger distance from the control greater than the threshold indicating a response. The graph 2310 of FIG. 13 shows specific growth rate distribution of MDA-MB-231 cells treated with 0.4 μM of doxorubicin has a much greater Hellinger distance from the control due to the depression of both the mean and standard deviation of the distribution. In some instances, ToR can be consistently quantified across conditions and cell types. With continued reference to FIG. 3D, the ToR is defined as a time point when the Hellinger distance crosses a threshold set by a maximum Hellinger distance of a solvent control from an untreated control, as shown by graphs 2400 of FIG. 14. Graphs A, B, and C of FIG. 14 show Hellinger distance over time between the DMSO treated cells and the untreated control showing that the maximum measured Hellinger distance is (Graph A) 0.112 for BT-474, (Graph B) 0.136 for MCF-7, (Graph C) and 0.092 to determine the threshold Hellinger distance used to determine ToR. For example, FIG. 3D is a graph 1315 of Hellinger distance versus time for determining probability distributions for 20 μM vinblastine and 0.016 μM docetaxel which quantifies the difference between each drug-treated group and the control to identify when the difference is significant enough to determine the ToR as shown by the threshold. The black dashed line represents the threshold determined by the maximum Hellinger distance between the DMSO and untreated control, the magenta dashed line shows the ToR for vinblastine, the maroon dashed line shows the ToR for docetaxel.

Often, the concentration tested just below the EC₅₀ elicits no response. For example, drugs that elicit no response never cross the Hellinger distance response threshold, as shown by the graphs 2400 in FIG. 14. Graphs D, E, and F of the graphs 2400 show conditions that show no response never cross the threshold because the Hellinger distance between the treated group and the control is similar to the Hellinger distance between the two controls.

In some instances, the QPI microscope system 110 uses the ToR at the tested concentration just above the calculated EC₅₀ as the nearest approximation of ToR at the calculated EC₅₀ (e.g., ToR at EC₅₀). Comparing ToR to DoR indicates that cytotoxic conditions elicit the fastest response, but even conditions classified as cytostatic often elicit a response in less than 24 h with a moderately negative relationship between ToR and DoR (R=−0.62, p=0.002), as illustrated in FIG. 3E. For example, FIG. 3E is a graph 1320 that illustrates that QPI data from the QPI microscope system 110 classifies each drug based on its cytotoxicity and how quickly it affects cell growth. The vertical dashed line is at DoR=1 as the threshold between a cytostatic and cytotoxic response. The horizontal dashed line is at ToR=24 h, as the division between fast and slow-acting drugs. Turning now to FIG. 3F, FIG. 3F is a graph 1325 plotting the ToR against the EC₅₀ further classifies drug-cell pairs based on sensitivity and speed of response. ToR near EC₅₀ plotted against the EC₅₀ classifies responses as fast or slow relative to drug sensitivity. The shape of each data point shows the cell line and the color describes a treatment therapy condition. FIG. 3H is a graph 1330 of normalized cell mass versus time for cancer cells exposed to treatment therapies.

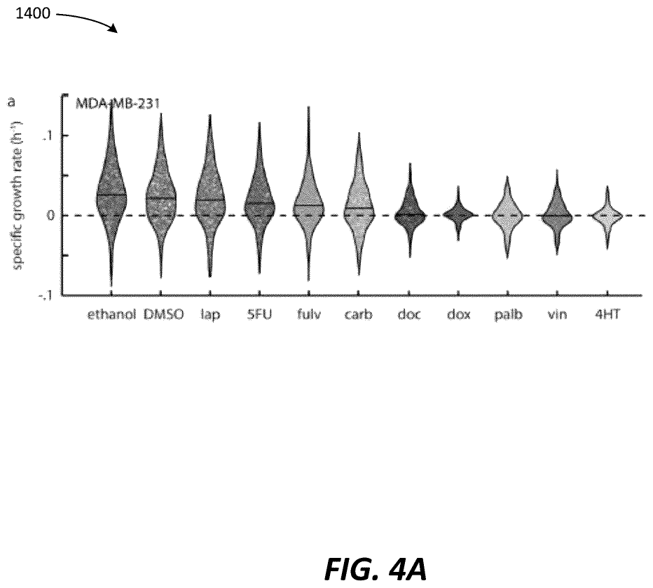

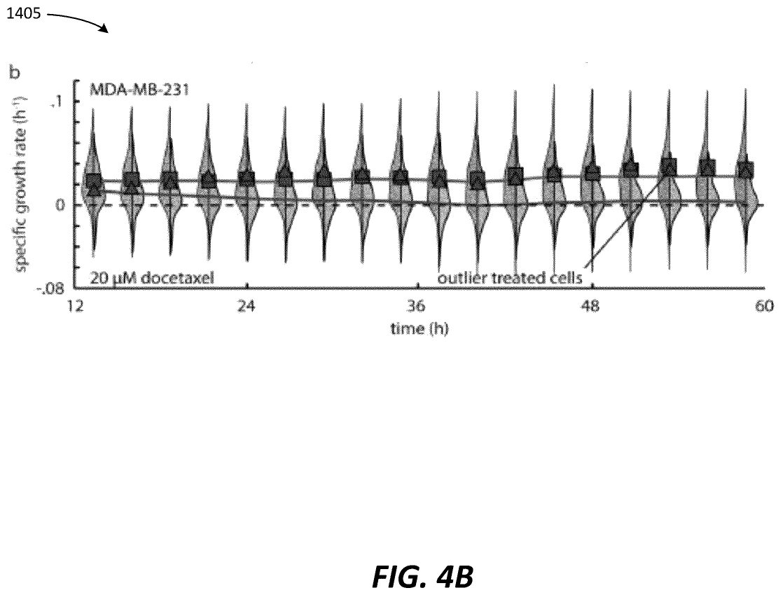

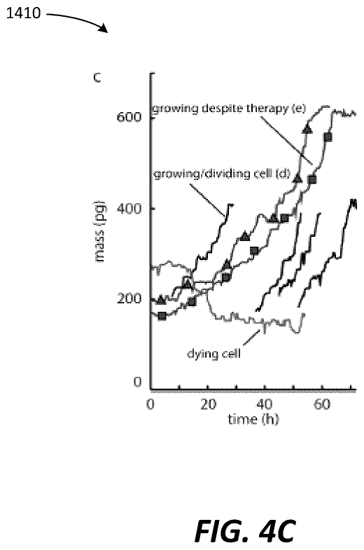

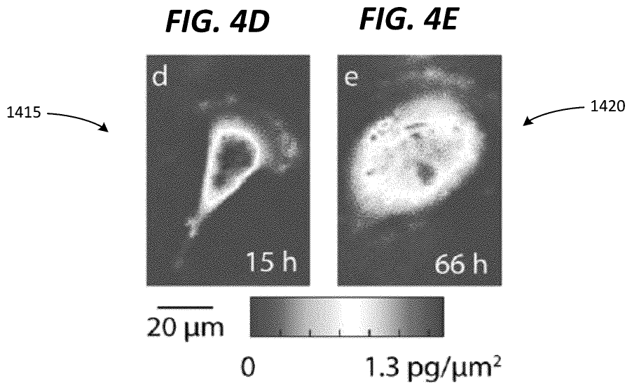

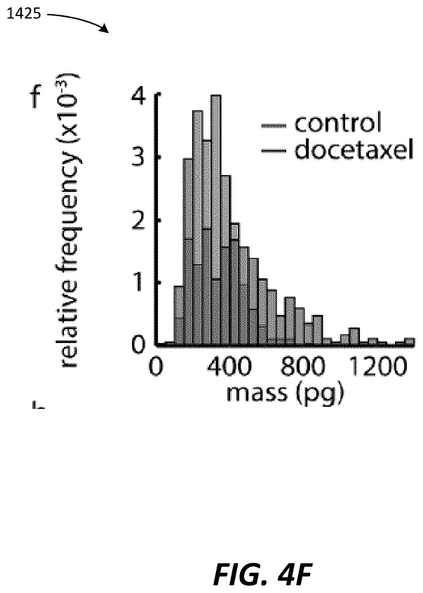

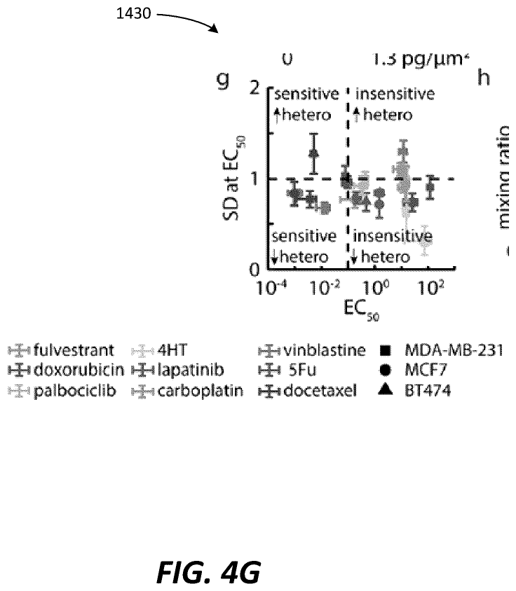

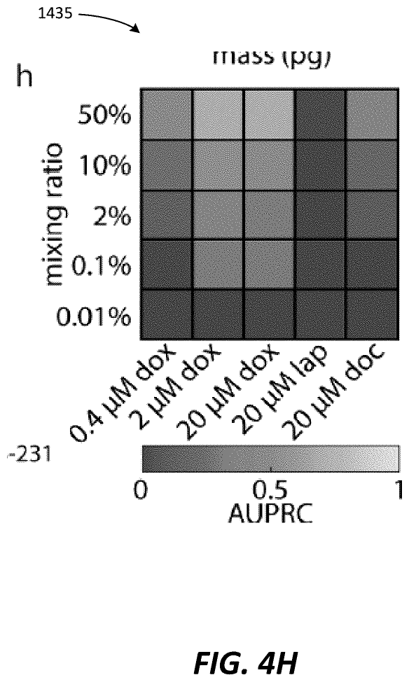

SGR in control populations shows intrinsic heterogeneity as shown by the large standard deviation in growth rates even in the control group, as shown by graphs 2500-2510 of FIG. 15. Graphs 2500-2510 show each cell line exhibits baseline growth heterogeneity in the control population. Mean shown as vertical solid line, mean+/−standard deviation shown as dashed lines. (Graph 2500: BT-474, n=20,593; Graph 2505: MCF-7, n=5,218; Graph 2510: MDA-MB-231, n=42,559). The intrinsic heterogeneity is impacted by treatment, with some drugs reducing heterogeneity by as much as fourfold, as in MDA-MB-231 with 20 μM doxorubicin as shown by graphs 2515 and 2520 of FIG. 15, with reference to FIG. 4A. Graph 2515 of FIG. 15 shows growth rate distribution of BT-474 cells exposed to 20 μM of each indicated drug. Graph 2520 of FIG. 15 shows growth rate distribution of MCF7 clusters exposed to 20 μM drug ordered from highest to lowest mean growth rate. Data is ordered from highest to lowest mean growth rate.

For example, FIG. 4A is a graph 1400 of growth rate distribution of 21,791 MDA-MB-231 cells at the end of 72 hours of 20 μM of indicated drug exposure. Additionally, cell-to-cell heterogeneity evolves over time during drug treatment, and heterogeneity change is captured by QPI using the QPI microscope system 110. For example, as illustrated in FIG. 4B, MDA-MB-231 cells (blue, n=1954 cells) treated with 20 μM docetaxel (maroon, n=1071 cells) show a gradual change in SGR distribution over 72 h with a distinctive long tail of non-responders that persists at 72 h, despite treatment with a cytostatic compound for >2.5 cell cycles. For example, FIG. 4B is a graph 1405 of MDA-MB-231 population growth rate distributions during 72 h treatment with DMSO and 20 μM docetaxel. Individual cell data is plotted and bound by the kernel density function of the distribution (black outline). Mean population response is shown as a horizontal line. The dashed line shows a growth rate of zero. Since QPI is based on longitudinal tracking of cells from microscopy images via the QPI microscope system 110 (described in greater detail below), individual non-responders are tracked backwards through the duration of the experiment. The graph 1405 of FIG. 4B shows two cells, with growth rates greater than mean of control (growth rate shown as square and triangle) at the end of experiment are traced back in time to determine how their growth rate evolved throughout the experiment. The dashed line shows a growth rate of zero.

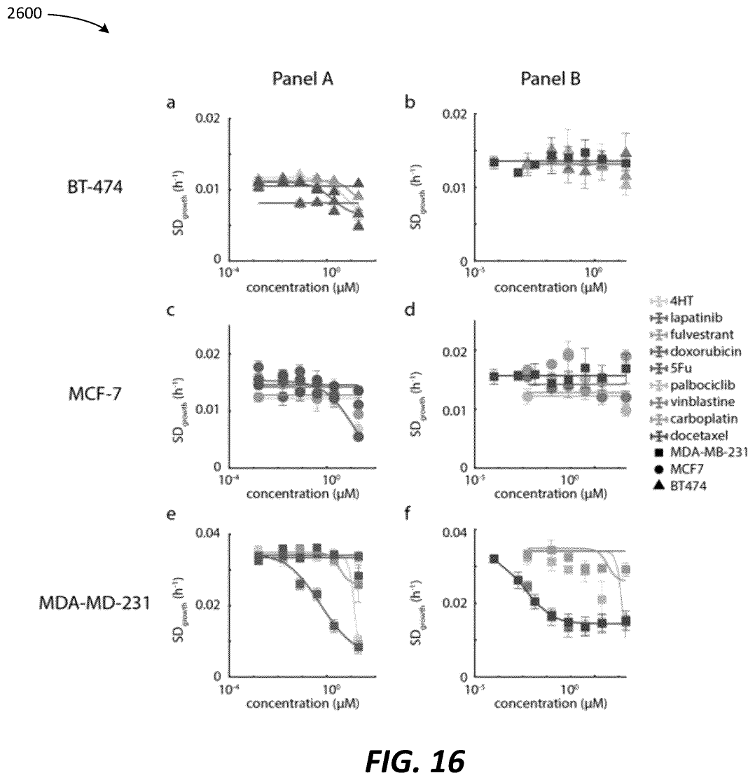

For example, as illustrated in FIG. 4C, select MDA-MB-231 cells treated with 20 μM docetaxel show growth that is distinctly different from the periodic doubling of cell mass observed in control populations. FIG. 4C is a graph 1410 of mass versus time tracks for two indicated cells of the graph 1405 of FIG. 4B. The select MDA-MB-231 cells treated with 20 μM docetaxel show growth in cells with a distinctively large mass as compared to control cells, as shown by FIGS. 4D-4F. Further, FIG. 4D is an image 1415 of a control cell from a cell panel indicating normal cell size and appearance. FIG. 4E is an image 1420 of a large cell from the cell panel persisting in the presence of 20 μM docetaxel. Referring back to FIG. 4C, given that docetaxel primarily acts as a microtubule inhibitor, QPI data from the QPI microscope system 110 may imply that treated cells, unable to divide or undergo apoptosis, reenter the cell cycle and continue accumulating mass at a similar rate as the control. The change in heterogeneity is dose-dependent, with an increase in dose corresponding to a reduction in cell-to-cell heterogeneity of growth, as shown by the graphs 2600 of FIG. 16. The graphs 2600 include plots that show standard deviation of 72 h cell cluster population specific growth rate as a function of drug concentration. Hill equation fit is only shown when there is a measurable response, as indicated when compared to a flat line fit using an F-test to reject the null hypothesis that the two fits are indistinguishable. Only a fraction (8 out of 18 conditions) show a measurable change in population standard deviation at increasing drug concentration. Error bars show SEM. Such impacts may be drug specific. In some instances, some compounds induce a significant decrease in the mean response, but no significant change to the spread (i.e., SD) within the population, even at high concentrations of therapy, such as in BT-474 treated with 20 μM docetaxel and 20 μM vinblastine. The graph 1410 of FIG. 4C shows that the MDA-MB-231 cells grew robustly throughout the experiment despite the high concentration of docetaxel. A dying cell from the experiment (gray) and normally growing cells from DMSO control (black) are also shown.

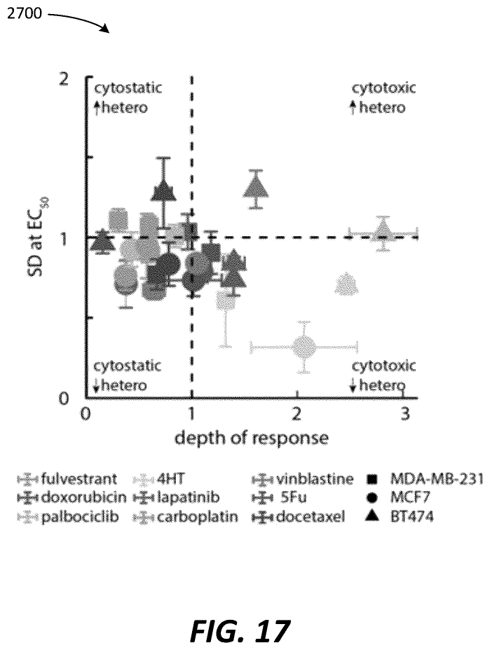

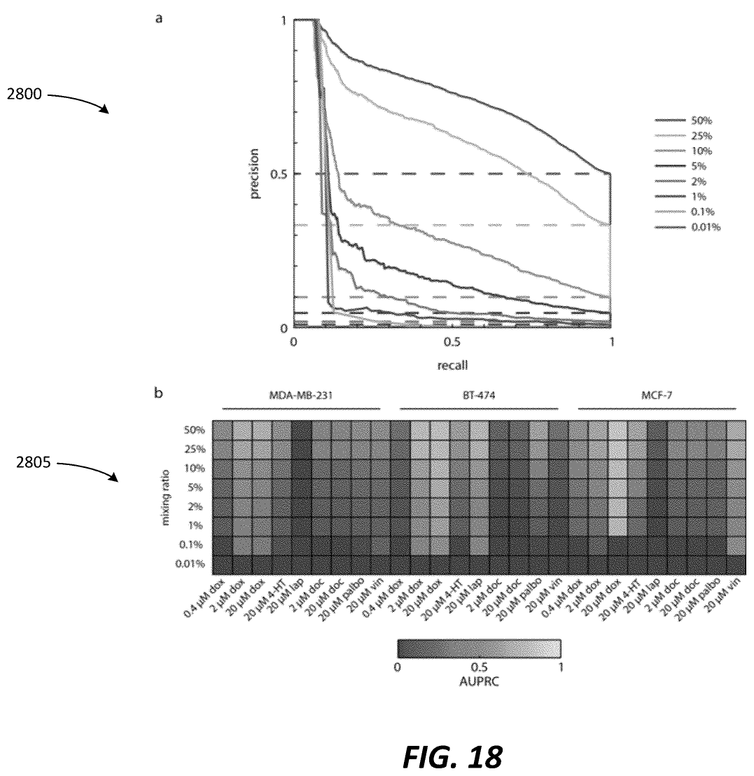

The SD is used at the tested concentration nearest the EC₅₀ (SD at EC₅₀) as a relevant measure of heterogeneity in a responding cell population. With reference to FIG. 4G, there is relationship between the measured heterogeneity during treatment (SD at EC₅₀) and EC₅₀ or ToR, indicating that the impact of drugs on growth heterogeneity provides a measurement of drug response that is independent of sensitivity and speed of response, as illustrated by FIG. 17. FIG. 17 is a graph 2700 that shows horizontal dashed line dividing fast responders from slow responders and vertical dashed line dividing cytotoxic conditions from cytostatic. Error bars show SEM. Heterogeneity is abbreviated as hetero. FIG. 4G is a graph 1430 of standard deviation at EC₅₀ plotted against EC₅₀ for cell growth. Points below horizontal dashed line (24 h) represent fast responders, and the vertical dashed line divides sensitivity from insensitivity of a cell line to a particular therapy. Heterogeneity is abbreviated as hetero. The limit of QPI is quantified to identify the proportion of resistant cells in an in silico mixture, using precision-recall analysis via the QPI microscope system 110, as shown by graph 2800 of FIG. 18. The graph 2800 shows precision-recall curves for MDA-MB-231 cells treated with 2 μM doxorubicin mixed with control cells in ratios of 50%, 25%, 10%, 5%, 2%, 1%, 0.1%, and 0.01%. Horizontal line for each ratio represents no ability to distinguish the control cells. Precision-recall curves have previously been shown to be an appropriate measure that is superior to receive operating characteristics for evaluating a classifier for unbalanced classes. This is especially important such cases where a fraction of resistant cells are considered that are small relative to the size of the population. With reference to FIG. 4H, the QPI microscope system 110 identifies resistant cells in proportions as low as 0.1-2%, however, this may be dependent on initial SGR, DoR, cell line, therapy, and concentration, as shown by graph 2805 of FIG. 18. The graph 2805 of FIG. 18 shows a heat map including a summary of precision-recall analysis for a range of responding conditions for all three cell lines. For example, FIG. 4H is a graph 1435 of normalized area under precision-recall curve (AUPRC) plotted against a percentage of control cells mixed into a drug-treated population (e.g., 0.4-20 μM doxorubicin, 20 μM lapatinib, 20 μM docetaxel).

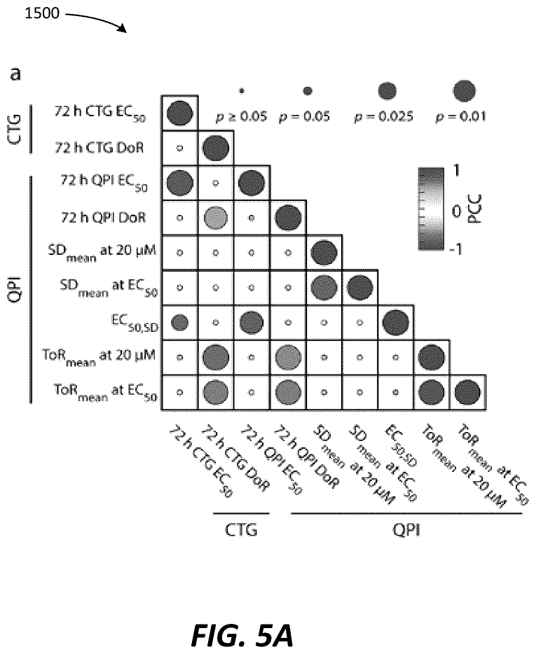

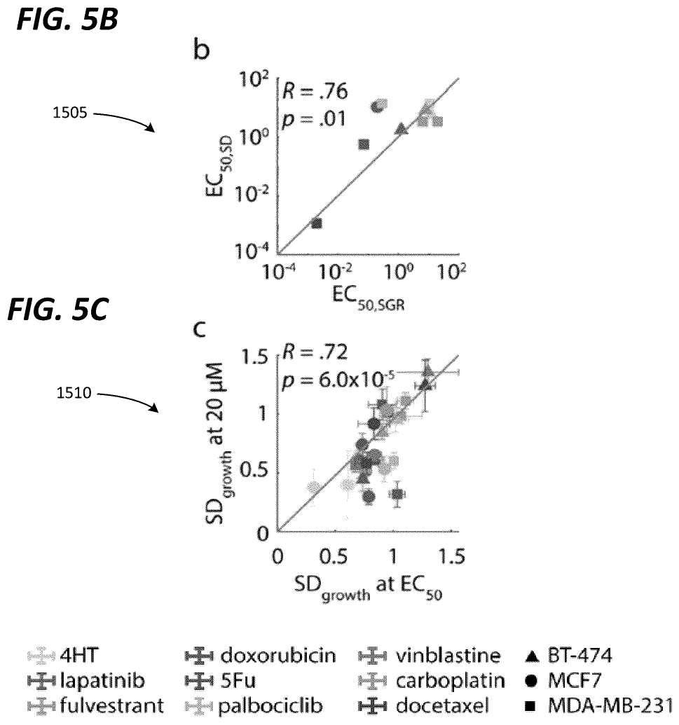

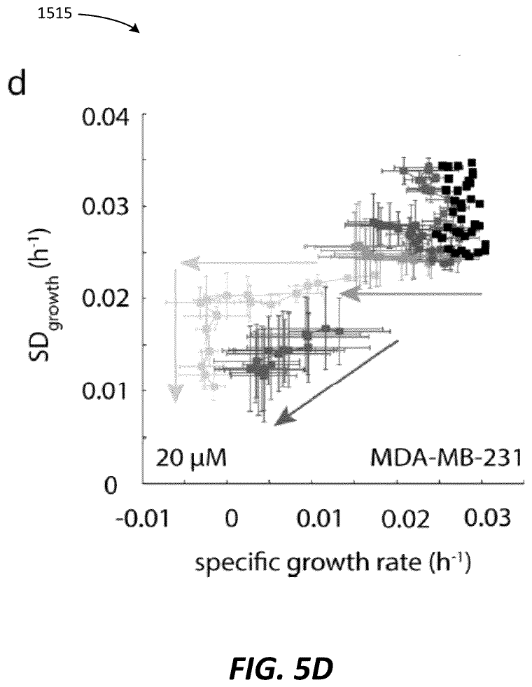

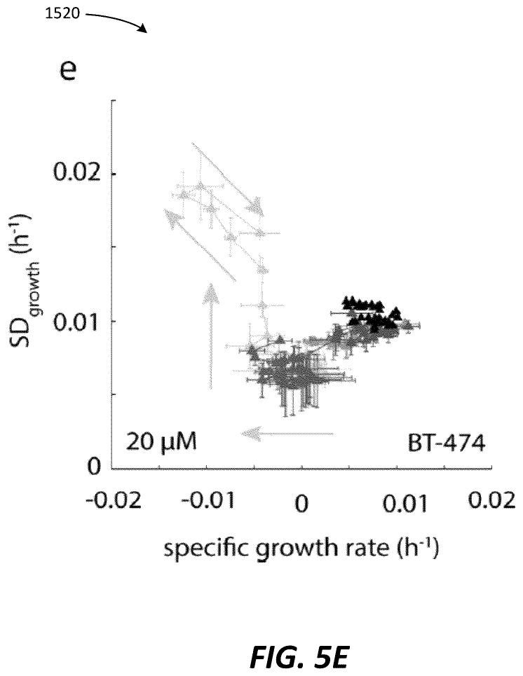

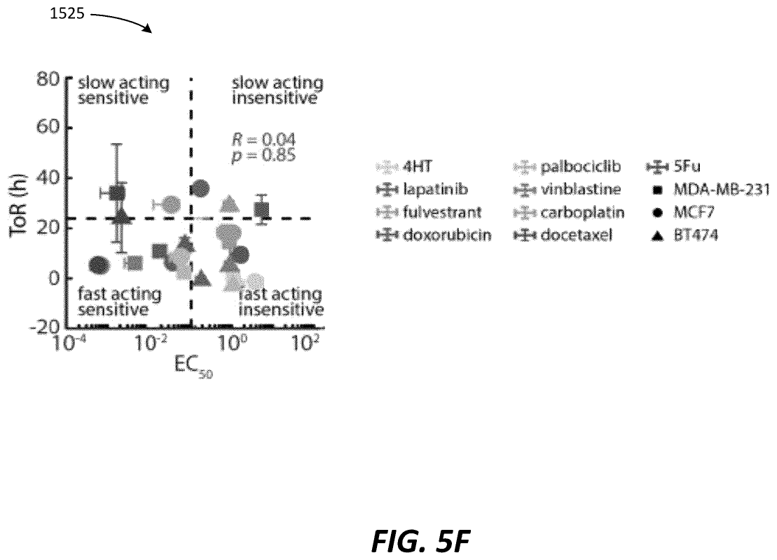

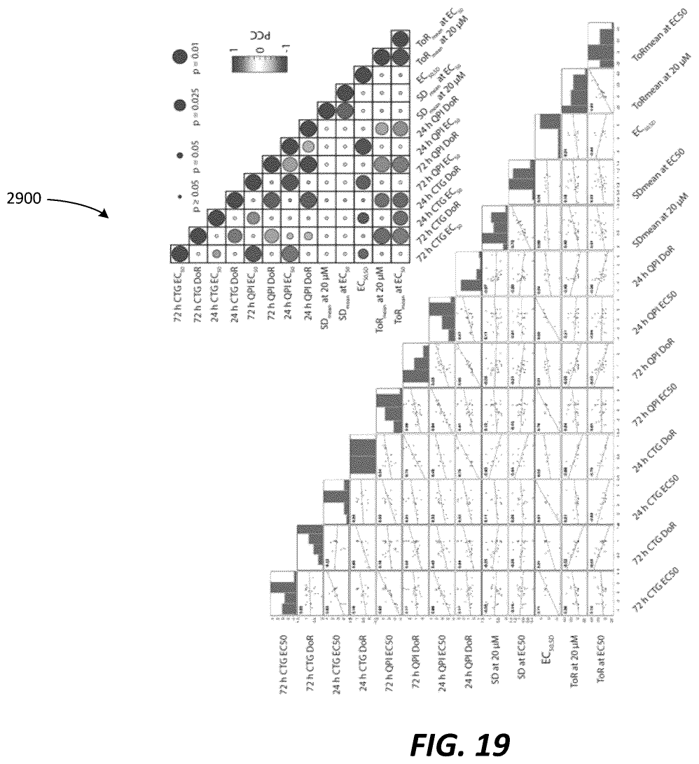

To further elucidate the relationships among QPI parameters, the QPI microscope system 110 determines the Pearson correlation coefficient between all QPI measured parameters (EC₅₀, DoR, ToR at EC₅₀, and SD at EC₅₀), as well as CTG-based EC₅₀ and DoR in FIG. 19, as shown by FIG. 5A. For example, FIG. 5A is a correlation matrix 1500 showing a relationship of functional measurements and predictive of cancer therapies affecting heterogeneity. The ToR at EC₅₀ has a moderate negative correlation to DoR (R=−0.62, p=0.002) indicating that more toxic drugs may cause a decrease in the time it takes to elicit a response. FIG. 19 is a correlation matrix 2900 that measures the Pearson correlation coefficient and shows strong correlations as dark blue, negative correlations as red, and no correlation as white. The size of the circle shows the level of significance of the measured Pearson coefficient. Correlation plots show the data used to determine the correlation coefficient. As illustrated by FIG. 5B, the EC₅₀ is determined for heterogeneity based on 4-parameter Hill equation fitting (FIG. 16) and is strongly correlated to the EC₅₀ measured for cell growth (R=0.76, p=0.01). Similarly, the SD at 20 μM is strongly correlated to the SD at EC₅₀ indicating that increased concentration beyond the EC₅₀ does not cause heterogeneity to decline beyond its level at the responding concentration (R=0.76, p<0.001), as illustrated in FIG. 5C. However, low correlations are shown between the change in heterogeneity, the ToR, and the EC₅₀. Furthermore, dimensional reduction using principal component analysis (PCA) suggests that EC₅₀ and DoR alone only account for about 70% of the information present in the data with the other 30% being split between SD and ToR. In some instances, EC₅₀, DoR, SD, and ToR derived from QPI provides mostly orthogonal measurements that independently describe different aspects of how cells respond to therapy (FIG. 19). FIG. 5B is a graph 1505 showing EC_{50,SD} correlated with EC_{50,SGR} and FIG. 5C is a graph 1510 showing standard deviation in growth at 20 μM concentration correlated with standard deviation of growth at EC₅₀

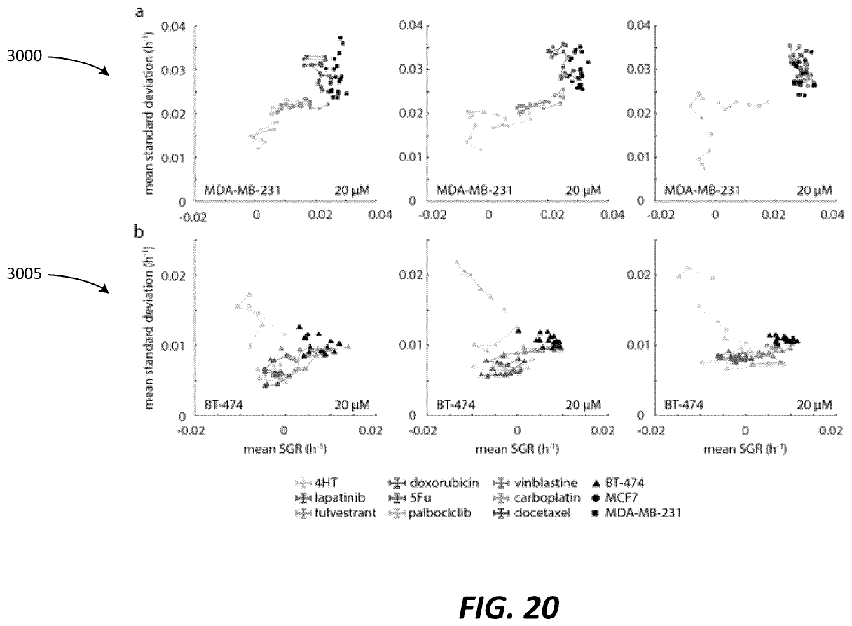

With reference to FIGS. 5D and 5E, to study how growth rate and heterogeneity change as a function of time, the QPI microscope system 110 parameterizes growth rate and heterogeneity by time and observed unique drug- and dose-dependent behaviors. For example, the change in heterogeneity found for both MDA-MB-231 and BT-474 cells occurs simultaneously with a change in growth rate, but when treated with 4HT, an estrogen receptor-targeted therapy that should affect ER-positive lines such as MCF-7, the behavior is quite different, as shown in graphs 3000 and 3005 of FIG. 20. The graph 3000 of FIG. 20 shows a plot of mean specific growth rate plotted against standard deviation in growth parameterized by time for MDA-MB-231 cells treated with a drug panel. Each track shows behavior from an individual experiment. The graph 3005 of FIG. 20 shows a plot of mean specific growth rate plotted against standard deviation in growth parameterized by time for BT-474 cells treated with a drug panel. Each track shows behavior from an individual experiment. Control cells are shown as a cluster of black points. As illustrated by FIG. 5D, MDA-MB-231 cells treated with 4HT experience a reduction in growth rate before heterogeneity is affected. For example, FIG. 5D is a graph 1515 of a plot of mean specific growth rate against standard deviation in growth parameterized by time for MDA-MB-231 cells treated with maximal concentration of a drug panel. Once the cells start dying, the heterogeneity of the population decreases dramatically at a constant growth rate.

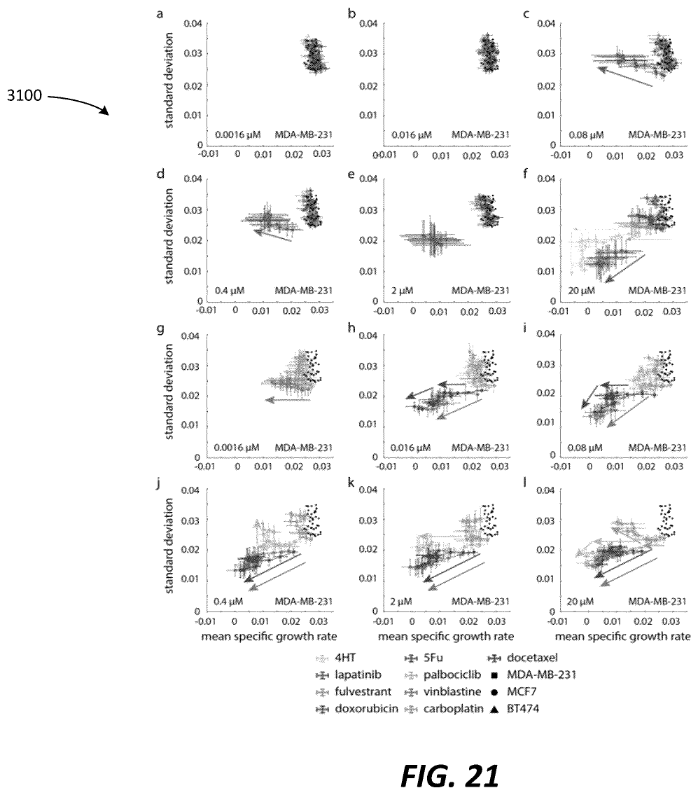

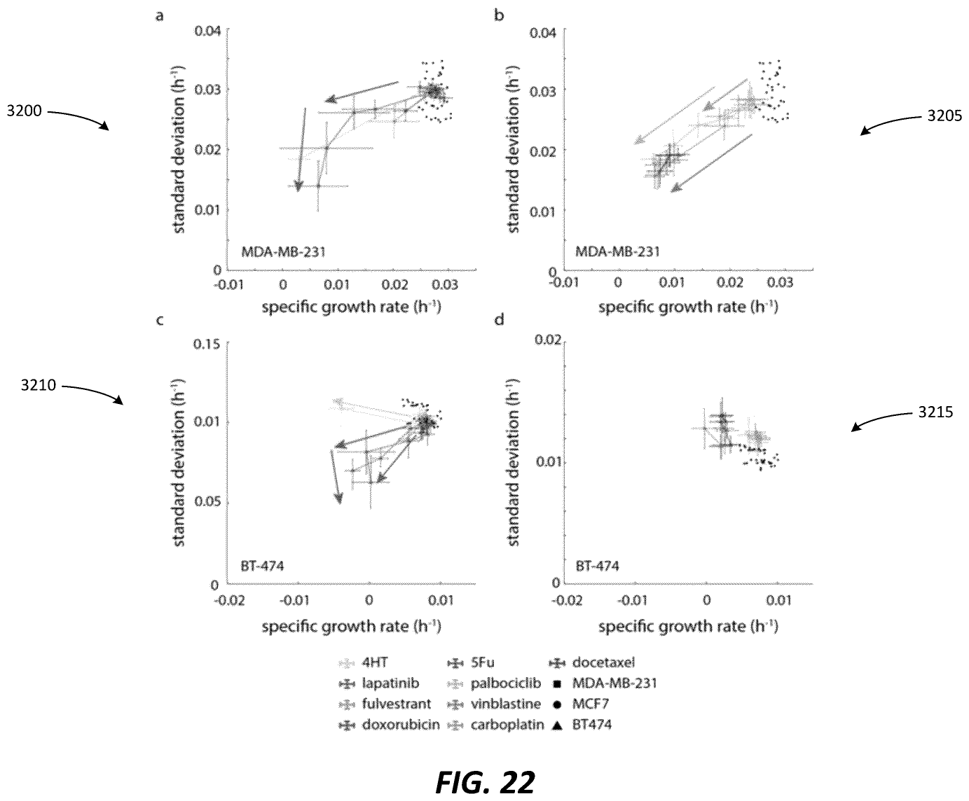

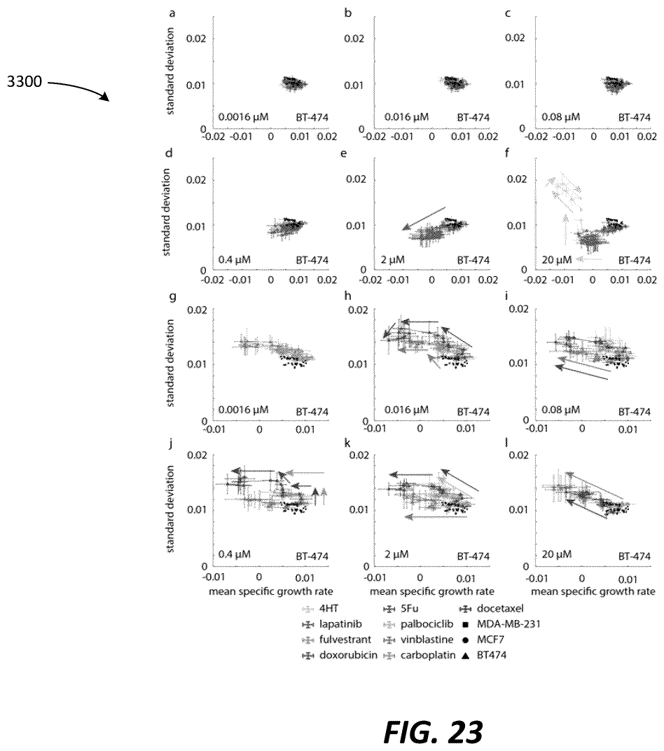

BT-474 cells however, as illustrated in FIG. 5E, increase in heterogeneity while their growth rate is decreasing. Generally, MDA-MB-231 cells respond to treatment with both reduced growth rate and reduced heterogeneity as shown in graphs 3100 of FIG. 21 and graphs 3200 and 3205 of FIG. 22, but BT-474 cells respond to treatment with both reduced growth rate and slightly increased heterogeneity for vinblastine, docetaxel, and 4HT as shown in graphs 3210 and 3215 of FIG. 22 and graphs 3300 of FIG. 23. For example, FIG. 5E is a graph 1520 of a plot of mean specific growth rate against standard deviation in growth parameterized by time for BT-474 cells treated with maximal concentration of a drug panel. Control cells for FIGS. 5D and 5E are shown as a cluster of black dots. Additionally, arrows in FIGS. 5D and 5E show forward direction in time. FIG. 5F is a graph 1525 of ToR versus EC₅₀ from QPI data from the QPI microscope system 110.

The graphs 3100 of FIG. 21 show graphs A and B of MDA-MB-231 cells treated with a drug panel show no change in growth rate or heterogeneity over time at the lowest concentrations. At middle concentrations in graphs C, D, and E of the the graphs 3100, doxorubicin shows a change in growth rate over time with little to no change in heterogeneity. Graph F of the graphs 3100 shows, at the highest concentration, doxorubicin simultaneously decreases in growth rate and heterogeneity, while 4HT first decreases in growth rate and then decreases in heterogeneity. Graphs G and H of the graphs 3100 show, at the lowest concentrations, vinblastine and docetaxel start at a growth rate similar to the control and growth rate decreases over time with little change in heterogeneity. Graph H of the graphs 3100 shows, at 0.08 M, MDA-MB-231 cells treated with both vinblastine and docetaxel decrease in growth rate and heterogeneity simultaneously. Graph J of the graphs 3100 shows, at 0.4 μM, MDA-MB-231 cells treated with vinblastine and docetaxel decrease in growth rate and heterogeneity simultaneously, but cells treated with palbociclib first decrease in growth rate with little change in heterogeneity relative to the control. Graph K of the graphs 3100 shows, at 2 μM, MDA-MB-231 cells treated with vinblastine and docetaxel decrease in growth rate and heterogeneity simultaneously, but cells treated with palbociclib decrease in growth rate with little change in heterogeneity. Graph L of the graphs 3100 shows, at 20 μM, MDA-MB-231 cells treated with vinblastine and docetaxel decrease in growth rate and heterogeneity simultaneously, but cells treated with palbociclib first decrease in growth rate before decreasing slightly in heterogeneity. Carboplatin causes a decrease in growth rate with little effect on heterogeneity relative to the control. Arrows show forward direction in time. Error bars show SEM.

The graph 3200 of FIG. 22 shows, as concentration increases, doxorubicin treated MDA-MB-231 cells decrease in both growth rate and heterogeneity. The graph 3205 of FIG. 22 shows, as concentration increases, palbociclib, carboplatin, and vinblastine treated MDA-MB-231 cells decrease in growth rate and heterogencity simultaneously. Graph 3210 of FIG. 22 shows BT-474 treated with doxorubicin, and lapatinib decrease in growth rate and heterogeneity, while 4HT treated cells decrease in growth rate at approximately constant standard deviation. Graph 3215 of FIG. 22 shows doxorubicin and vinblastine treated BT-474 cells decrease in growth rate as concentration increases while heterogeneity remains constant. Arrows show direction of increasing concentration. Error bars show SEM.

Graphs A, B, C, and D of the graphs 3300 of FIG. 23 show, at concentrations below the EC₅₀, both standard deviation and the mean specific growth rate for drug treated populations are indistinguishable from the control (shown as black dots). Graph E of the graphs 3300 show, near the EC₅₀ concentration, the mean growth rate and heterogeneity of the population both decrease simultaneously. In contrast, Graph F of the graphs 3300 shows BT-474 cells respond to 4-hydroxy-tamoxifen by first experiencing reduced growth rate, and then a large spike in heterogeneity in response to the therapy. Only after the growth rate is further reduced does the heterogencity of the population begin to decrease. Graph G of the graphs 3300 shows BT-474 responds to vinblastine at the lowest concentration (e.g., 0.0016 μM) causing a decrease in growth rate at constant heterogeneity that is greater than the control. Graph H of the graphs 3300 shows BT-474 responds to both vinblastine and docetaxel at 0.016 μM causing a decreased growth rate and increased heterogeneity. Graph I of the graphs 3300 shows, at a concentration of 0.08 μM, BT-474 cells respond with reduced growth rate and increased heterogeneity. Graph J of the graphs 3300 shows, when treated with 0.4 μM of therapy, BT-474 cells respond to docetaxel with reduced growth rate and increased heterogeneity but respond to vinblastine with reduced growth rate at the same level of heterogeneity as the control. Graph K of the graphs 3300 shows at 2 μM of treatment concentration population heterogeneity is increased and growth rate decreases in response to both docetaxel and palbociclib. Graph L of the graphs 3300 shows, at 20 μM concentration, the growth rate of docetaxel and vinblastine treated cells decreases, while the heterogeneity for both populations increases. Arrows show forward direction in time. Error bars show SEM.

The application of QPI with the QPI microscope system 110 is a multiparametric, label-free, high-throughput tool for measuring growth response of adherent cells to cancer therapies. QPI predictions of which drugs a given population of cells will not respond to and the concentration at which cells demonstrate sensitivity to therapy (EC₅₀) is strongly concordant with traditional CTG measurements. Additionally, QPI with the QPI microscope system 110 includes additional metrics for characterization of drug response at a single cell level. The DoR measured using QPI with the QPI microscope system 110 is a useful tool for classifying the effect of therapies as either cytostatic or cytotoxic. As a method of tracking growth rates over time, the QPI microscope system 110 measures response dynamics of single cells, including ToR and heterogeneity, and includes tracking of outliers. Considering all of the noted parameters (e.g., EC₅₀, DOR, ToR at EC₅₀, and SD at EC₅₀) shows orthogonality and the dynamic responses of populations over time. A summary of the measured parameters for all studied conditions is included in Table 9 below.

TABLE 9
Summary of QPI Measured Parameters.

| | Cell | | | | | | | | | |
|---|---|---|---|---|---|---|---|---|---|---|
| Drug Name | Line | EC_{50, CTG} | DoR_{CTG} | EC_{50, QPI} | DoR_{QPI} | SD_{20 μM} | SD_{EC50} | EC_{50, SD} | ToR_{20 μM} | ToR_{EC50} |
| 4HT | BT-474 | 4.2 | 1.0 | 18.0 | 2.5 | 0.6 | 0.7 | 1.7 | −0.9815 | −0.9815 |
| 4HT | MCF-7 | 3.9 | 1.0 | 75.2 | 2.1 | 0.3 | 0.3 | NaN | −1.3078 | −1.3 |
| 4HT | MDA- | 6.9 | 1.0 | 15.1 | 1.3 | 0.3 | 0.6 | 2.7 | 1.0 | 1.0 |
| | MB-231 | | | | | | | | | |
| Lapatinib | BT-474 | 1.7 | 0.6 | 1.6 | 1.4 | 0.6 | 0.8 | 0.5 | −1.0616 | −0.9 |
| Lapatinib | MCF-7 | NaN | NaN | 1.5 | 0.4 | 0.5 | 0.7 | NaN | 38.2 | 35.8 |
| Lapatinib | MDA- | NaN | NaN | NaN | NaN | 1.1 | NaN | NaN | NaN | NaN |
| | MB-231 | | | | | | | | | |
| Fulvestrant | BT-474 | 1342.8 | 0.6 | 11.6 | 0.6 | 0.8 | 0.9 | 2.1 | 28.9 | 28.9 |
| Fulvestrant | MCF-7 | 0.5 | 0.3 | 0.2 | 0.4 | 0.4 | 0.8 | NaN | 23.2 | NaN |
| Fulvestrant | MDA- | 31.4 | 1.0 | 11.4 | 0.6 | 0.9 | 1.1 | 1.2 | 14.4 | 14.4 |
| | MB-231 | | | | | | | | | |
| Doxorubicin | BT-474 | 0.4 | 0.5 | 0.5 | 1.4 | 0.4 | 0.7 | NaN | 3.4 | 12.9 |
| Doxorubicin | MCF-7 | 0.2 | 1.0 | 0.2 | 1.1 | 0.3 | 0.8 | 2.4 | −1.3 | 6.4 |
| Doxorubicin | MDA- | 0.3 | 1.0 | 0.1 | 1.0 | 0.3 | 1.0 | −0.6 | −3.0 | 10.8 |
| | MB-231 | | | | | | | | | |
| 5FU | BT-474 | 0.1 | 0.1 | 0.1 | 0.2 | 0.9 | 1.0 | NaN | NaN | NaN |
| 5FU | MCF-7 | 10.8 | 0.9 | 25.7 | 1.0 | 0.6 | 0.7 | NaN | 9.4 | 9.4 |
| 5FU | MDA- | 6.0 | 0.5 | 118.0 | 1.2 | 0.9 | 0.9 | NaN | 15.2 | 15.2 |
| | MB-231 | | | | | | | | | |
| Palbociclib | BT-474 | 0.0 | 0.9 | 11.8 | 1.6 | 1.2 | 1.3 | NaN | −3.0 | −3.0 |
| Palbociclib | MCF-7 | 0.0 | 1.0 | 0.0 | 1.0 | 0.6 | 0.8 | NaN | 4.4 | 8.5 |
| Palbociclib | MDA- | 0.0 | 0.7 | 0.0 | 0.6 | 0.5 | 0.7 | NaN | 1.6 | 2.4 |
| | MB-231 | | | | | | | | | |
| Vinblastine | BT-474 | 6.4 | 1.0 | 14.9 | 2.8 | 0.9 | 1.0 | NaN | 4.9 | 4.9 |
| Vinblastine | MCF-7 | 1.6 | 0.8 | 0.3 | 0.4 | 0.5 | 0.9 | NaN | −3.0 | 6.4 |
| Vinblastine | MDA- | 3.0 | 1.0 | 0.4 | 0.8 | 0.5 | 1.0 | 2.7 | 4.7 | 6.1 |
| | MB-231 | | | | | | | | | |
| Carboplatin | BT-474 | NaN | NaN | NaN | NaN | 1.0 | NaN | NaN | 38.7 | 38.7 |
| Carboplatin | MCF-7 | 15.5 | 0.4 | 14.2 | 0.6 | 0.9 | 0.9 | NaN | 18.1 | 18.1 |
| Carboplatin | MDA- | NaN | NaN | 9.1 | 0.3 | 1.0 | 1.1 | 1.2 | 18.3 | 18.3 |
| | MB-231 | | | | | | | | | |
| Docetaxel | BT-474 | 0.0 | 0.6 | 0.0 | 0.8 | 0.8 | 0.8 | NaN | 10.9 | 24.1 |
| Docetaxel | MCF-7 | −3.3 | 0.6 | −7.0 | 0.8 | 0.8 | 0.8 | NaN | 5.9 | 5.3 |
| Docetaxel | MDA- | 0.0 | 0.7 | 0.0 | 0.7 | 0.5 | 0.8 | −6.8 | 3.5 | 33.9 |
| | MB-231 | | | | | | | | | |

In some instances, the QPI microscope system 110 measures time-averaged growth rate for individual cells/clusters, both on a scale of the entire experiment and the growth rate over smaller time intervals demonstrating how QPI tracks the temporal dynamics of growth and heterogeneity (e.g., as shown in FIGS. 3C, 5D, and 5E). With reference back to FIG. 4B, the graph 1405 shows QPI traces cells back through the assay to determine whether the cells were intrinsically resistant or recovered from the initial growth inhibition by adapting to its presence in the environment. Both MCF-7 cells and BT-474 cells may be sensitive to lapatinib, a therapy targeting the HER2 receptor, with an EC₅₀ of ˜1.5 μM. Although the sensitivity to lapatinib for the two cell lines is very similar, BT-474, a HER2+ cell line, shows a cytotoxic response, a decrease in heterogeneity by 30% relative to MCF-7, and a ToR that is 60 h faster than the cytostatic response of MCF-7. In some instances, it may be difficult for QPI to distinguish resistant cells in the treated MCF-7 population, however, the QPI microscope system 110 identifies control cells mixed into the treated BT-474 population down to a mixing ratio of 0.1% (FIG. 18). The multiple parameters together demonstrate how much more effective lapatinib is against BT-474 than MCF-7 cells than sensitivity alone. In some instances, this level of insight is useful for understanding how resistant cells and subpopulations develop and for identifying therapies that overcome resistance.

In some embodiments, several aspects of multiparametric QPI point towards clinical applications, in addition to its concordance with CTG, which is widely used in clinical trials of functional oncology. QPI includes a readout of cell response and is marker free. In addition, QPI is not susceptible to drug-stain interference or false positives from sub-lethal/sub-cytostatic alterations in ATP production. QPI uses relatively few cells, making it amenable for application to clinical samples with limited cell numbers or where expansion is difficult. The examples and embodiments described herein use approximately 200,000 cells to achieve sufficient density for imaging in each of 96-wells of the well-plate 105, with the QPI microscope system 110 measuring the response of up to half of the total cells plated in the well-plate 105. In some embodiments, a different number of cells may be used and the well-plate 105 may include a different number of wells.

In some instances, 72 h QPI experiments are not required for measuring cell sensitivity to therapy. In most instances, 24 h QPI is sufficient to predict treatment response relative to CTG and 24 h QPI results are strongly correlated with 72 h QPI results (FIG. 11). The correlation is consistent with ToR data showing that 16 conditions elicite a response near the EC₅₀ concentration in less than 24 h. Taken together, QPI may be applied to classify drug responses as either fast or slow responders based on ToR and QPI data from the QPI microscope system 110 can be used to streamline testing of clinical samples. As part of a clinical workflow, plating samples on two or more plates and starting all drug treatments at the same time after plating is common. The QPI microscope system 110 images fast responders for the first interval (e.g., 24 h) and slow responders for the next period (e.g., 24-48 h). The QPI microscope system 110 applies QPI to rapidly quantify drug responses to a larger panel of drugs with varying mechanisms in as little as 48 h. Overall, the rich, quantitative data on cell responses measured by QPI can generate new insights into drug response that may ultimately inform clinical decision-making.

Referring to FIG. 1A and in accordance with the embodiments described herein, the QPI microscope system 110 performs QPI with a differential phase contrast (DPC) microscope (e.g., a QPI microscope 120). For example, the QPI microscope system 110 includes the QPI microscope 120. In some instances, the QPI microscope 120 includes the components in FIG. 1A and may be placed inside a cell culture incubator for temperature, humidity, and 5% CO2 control. As illustrated in the embodiment of FIG. 1A, the QPI microscope 120 includes a microscope body (e.g., a housing) 125. The microscope body 125 includes and/or couples to the components of the QPI microscope 120, described below. The QPI microscope 120 captures images of cells in each well of the well-plate 105 using, for example, a 10×, 0.25 numerical aperture (NAobj) objective (e.g., an objective 130) and a Grasshopper3 USB camera (e.g., a camera 135) including 1920×1200 pixels that are, for example, 0.54 μM in size (e.g., manufactured by Teledyne FLIR, Wilsonville, OR). In some embodiments, the objective 130 is configured to magnify the image captured by the camera 135. In some embodiments, the QPI microscope 120 includes a lens 170 and a mirror 175. In some embodiments, the lens 170 is a plurality of lenses. When capturing an image of a plurality of therapy treatment samples with the camera 135, the mirror 175 reflects light emitted from the LED array 150 such that the therapy treatment samples are visible to the camera 135 to render an image. The lens 170 receives reflected light from the mirror 175 to be captured by the camera 135. In some embodiments, the lens 170 is configured to focus the camera 135 on the plurality of treatment samples. In some embodiments, the lens 170 magnifies the image captured by the camera 135. In some embodiments, an exposure time of the camera 135 is set to 50 μs and gain is 25 dB. In some embodiments, focus may be maintained with an automated focusing algorithm for the camera 135. In some embodiments, the QPI microscope 120 includes a focus adjustment mechanism 140. In some embodiments, the focus adjustment mechanism 140 receives a user input indicative of a desired focus level of the camera 135. In response to receiving the user input, the focus adjustment mechanism 140 may manually adjust the focus of the camera 135 such that the camera 135 focuses on the well-plate 105. In some embodiments, the camera 135 is configured to capture images of a plurality of samples (e.g., therapy treatment samples as described below) over a period of time using an amount of illumination from the LED array 150.

In some embodiments, the QPI microscope 120 is coupled to a high-speed xy translation stage (e.g., a translation stage) 145 (e.g., MLS203, manufactured by Thorlabs, U.S.A), set to, for example, an acceleration of 2480 mm/s2 and a maximum velocity of 400 mm/s, to image each location of the well-plate 105 with a temporal resolution of 20 minutes. In some embodiments, the translation stage 145 is configured to hold and move the well-plate 105 having a plurality of samples (e.g., a plurality of therapy treatment samples). In some embodiments, for illumination, the QPI microscope 120 includes an 18 mm square 8×8 light emitting diode (LED) array (e.g., an LED array 150) including a plurality of LEDs, positioned approximately 24 mm above a sample plane of the well-plate 105 for a numerical aperture of illumination (NALED array) of 0.39. In some embodiments, the LED array 150 is configured to illuminate the well-plate 105 including the plurality of samples. In some embodiments, a coherence parameter, denoted as σ, which is a ratio of NALED array: NAobj is 1.5235,36. The LED array 150 is controlled, for example, via an Arduino Metro M4 (e.g., manufactured by Adafruit, U.S.A.) (e.g., an electronic controller 155). In some embodiments, the LED array 150, the translation stage 145, and the camera 135 are each electronically (e.g., communicatively) connected to the electronic controller 155. In other words, the electronic controller 155 may control an operation of each of the LED array 150, the translation stage 145, and the camera 135. For example, the electronic controller 155 transmits a light signal indicative of an illumination level to the LED array 150 and the LED array 150 illuminates the well-plate 105 based on the light signal.

In some embodiments, the electronic controller 155 transmits a movement signal to the translation stage 145 and the translation stage 145 moves the well-plate 105 based on the movement signal. In some embodiments, the electronic controller 155 is electronically connected to a motor 160. The motor 160 is coupled to a drive shaft 165 and configured to drive the drive shaft 165. The drive shaft 165 is coupled to the translation stage 145 and configured to move the translation stage 145. In some embodiments, the electronic controller 155 transmits the movement signal to the motor 160 and the motor 160 drives the drive shaft 165 to move the translation stage 145 (holding the well-plate 105 having the plurality of therapy treatment samples) based on the movement signal. In some embodiments, the electronic controller 155 transmits an image signal to the camera 135 and the camera 135 captures images of the plurality of samples on the well-plate 105 based on the image signal. In some embodiments, the QPI microscope system 110 includes the electronic controller 155 and the electronic controller 155 may be electronically connected to each of the components of the QPI microscope 120. It should be understood that the electronic controller 155 may be configured to calculate or determine values for each of the representations illustrated by FIGS. 1B-27, as described in greater detail below.

In some embodiments, the electronic controller 155 implements DPC image acquisition and phase retrieval with the QPI microscope 120. In some examples, the QPI microscope 120 captures, via the camera 135, four images with half circle illumination (top, bottom, left, right), via the LED array 150, in less than one second including QPI microscope motion. For example, the electronic controller 155 receives a plurality of images of the plurality of treatment samples from the camera 135 over the period of time. Opposing pairs of images are used to compute the phase gradient in two orthogonal directions, via the electronic controller 155. The QPI microscope 120 computes, via the electronic controller 155, a phase shift of the captured images by deconvolution with an estimated optical transfer function via Tikhonov regularization. In some embodiments, parameters for obtaining phase reconstruction include illumination angles (e.g., 90 and 180 degrees because QPI images are taken along axes normal to each other) and a regularization parameter (e.g., 1×10-3), which is determined experimentally based on system calibration (described below).

The QPI microscope system 110 controls for the effect of drug solvents and phototoxicity using on-plate solvent controls during each experiment. In some embodiments, the on-plate solvent controls are matched to the highest concentrations of solvent used in the experiment (e.g., 0.125%). For example, as illustrated by FIG. 1A, both plates include DMSO controls as most compounds are solubilized in DMSO, as illustrated by a plate map 180 of the well-plate 105. The plate map 180 shows how therapies for a drug panel are organized. For example, Docetaxel uses an expanded range of concentrations to capture the typically low EC₅₀ for a drug, noted in maroon below the plate map 180. Ethanol control wells may also be used on the well-plate 105 as 4-hydroxy-tamoxifen is solubilized in ethanol. Additionally, the power of the LED array 150 at a sample plane may be ˜790 nW integrated over a single field of view corresponding to a flux of 4×106 photons per μm2. In some embodiments, the power of the LED array 150 may be less than the 5×108 photons per μm2 to be considered a safe exposure, and at a longer wavelength, 624 nm in embodiments described herein versus 473 nm, and, consequently, lower energy per photon. Referring back to the graph 1425 of FIG. 4F, comparison among solvent controls, untreated controls, and cell counting performed on replicate plates (under no illumination) may yield no significant difference in control growth rate. For example, the graph 1425 of FIG. 4F is a histogram showing a final mass for docetaxel treated cells (red, n=343 cells) and DMSO treated cells (blue, n=563).

The QPI microscope 120 is calibrated prior to imaging, to validate the QPI microscope system 110 and to ensure the measurements are not impacted by misalignment. In some embodiments, a first calibration experiment validates centering of the LED array 150 by comparing a raw intensity from each half of the LED array 150 while imaging an empty sample of the well-plate 105. In some embodiments, after centering and aligning the LED array 150, the intensity from each half of the LED array 150 is equal to about 5% of the mean for all four half circles, as illustrated in a graph 1600 of FIG. 6. For example, the graph 1600 shows mean intensity data for the LED array 150 with each half of the LED array 150 illuminated as during cell growth experiments, error bars represent the standard deviation of intensity. Dashed green line is plotted at an intensity of 43,400 a.u., the mean intensity of all 4 halves of the LED array 150. The QPI microscope system 110 calibrates, using the electronic controller 155, phase measurements by imaging polystyrene microbeads (e.g, manufactured by Polysciences, Warrington, PA), a common calibration standard for QPI microscopes, such as the QPI microscope 120. In some embodiments, to prepare a bead sample, bead stock is diluted by a factor of 10 such that a bead solution is about ˜0.25% microbeads (weight/volume). The bead solution is mixed using a vortex for 10 seconds and then pipetted 100 μL onto a glass slide. After the water evaporates, pipette 50 μL of NOA73 (e.g., Norland Optical Adhesives, n=1.56) on top of the beads (n=1.583) and flatten the polymer with a coverslip to obtain an even layer of polymer. In some embodiments, the bead is cured for 1 minute in a UV oven to obtain a sample with a difference in refractive index comparable to that between cells and cell culture media (e.g., An=0.023). In some embodiments, the QPI microscope 120 images the beads and the mean refractive index of the beads is ˜1.584, within 0.1% of previously reported values of 1.583 for 624 nm light, as illustrated by an image 1605 of FIG. 6. The image 1605 shows example images of polystyrene beads embedded in NOA73 used to calibrate phase shift measurements. In some embodiments, the QPI microscope 120 images the beads 100 times in a 3-minute interval to measure the temporal precision, and, in some instances, the temporal coefficient of variation is 5.4%, as illustrated by a graph 1610 of FIG. 6. The graph 1610 shows measured refractive index of polystyrene beads embedded in NOA73 during repeated QPI measurements over time. Blue line shows the mean refractive index measured across all the beads (n=20), error bars show the standard error of the mean. Black line is at n=1.583, the previously reported refractive index for polystyrene, and red lines show the uncertainty in the known refractive index of the NOA73 matrix.

In some embodiments, the QPI microscope 120 segments the cells using a Sobel filter, via the electronic controller 155, to find cell edges, and morphological operators to create a mask. Single MDA-MB-231 cells can be further segmented using a watershed algorithm. In some embodiments, cells are masked, using the electronic controller 155, and an 8th order polynomial fit is removed from the background prior to averaging images, using the electronic controller 155, from each experiment to correct for aberrations and optical artifacts. A rolling ball filter, using, for example, a disk structuring element of 100 px, is applied by the electronic controller 155 to remove high spatial frequency noise. In some embodiments, the electronic controller 155 determines cell mass using a cell average specific refractive increment of 1.8×10-4m3/kg23. For example, the electronic controller 155 determines cell mass for each therapy treatment sample of the plurality of treatment samples based on each image of the plurality of images received from the camera 135. The QPI microscope 120 tracks, using the electronic controller 155, segmented cells from frame to frame of captured images based on approximate minimization of the distance between cell objects in successive frames in terms of cell mass and position in x and y directions. For example, the electronic controller 155 tracks cell mass over the period of time for each therapy treatment sample of the plurality of treatment samples.

Prior to operation of the QPI microscope 120, drugs (e.g., treatment therapies) are mixed with a volume of solvent to make a 20 mM stock solution. In some instances, stock solutions are stored in a −20° C. freezer and are not thawed more than five times to preserve the efficacy of the treatment therapies. A stock concentration of the treatment therapies is aliquoted into media to make a 40 μM solution, which is serially diluted on a 96-well plate (e.g., the well-plate 105) with 1 mL wells to make a solution that is double a concentration for the assay. In some instances, the diluted therapies are added to cells 3 h prior to the start of the assay at a 1:1 ratio of drugged media to cell media, to dilute the concentration of therapy to its final concentration.

In some instances, 1500 cells are plated in each well of two 96 well plates (e.g., two well-plates 105) with 100 μL of media to allow space for drugged media to be added. Cells may be incubated in cell culture conditions for 18 h prior to dosing. In some instances, 100 μL of diluted treatment therapies and solvent controls are added to the cells 3 h prior to the start of imaging. Cells may be allowed to incubate on the QPI microscope 120 in cell culture conditions (e.g., 37° C. and 5% CO2) for an hour prior to focusing the center of each well of the well-plate 105. In some embodiments, the electronic controller 155 selects nine imaging positions per well and each position is imaged every 20 minutes via the QPI microscope 120 with a single autofocus before each imaging cycle to account for thermal and z-stage drift. After 24 h of imaging a first well-plate 105, a first CTG assay is performed on a second well-plate 105 (e.g., FIG. 1B) while continuing to image the first well-plate 105 for a total of 72 h. After 72 h of imaging, the second CTG assay is performed on the first well-plate 105.

In some embodiments, the electronic controller 155 determines a plurality of response parameters for each therapy treatment sample of the plurality of treatment samples based on the cell mass over the period of time. In some instances, the electronic controller 155 performs cell counting experiments by measuring proliferation throughout the duration of an experiment. For example, the electronic controller 155 counts cells in three different ethanol-treated wells of the well-plate 105 at 0 h, 18 h, 36 h, 48 h, and 72 h to measure the doubling time for MCF-7 and MDA-MB-231 cells. The following equation (Equation 1) may be used to compute the exponential growth constant for each cell line:

$$SGR = \frac{ln ( 2 )}{{t}_{doubling}} \qquad Equation 1$$

Such that t_{doubling} is the doubling time measured using cell counting for each cell line, ln(2) is the natural logarithm of 2, and SGR is the specific growth rate also known as the exponential growth constant.

In some instances, cell lines are acquired from the American Type Culture Collection (ATCC) and routinely screened for mycoplasma infection using, for example, the Agilent MycoSensor qPCR assay. In some embodiments, MCF-7 cells are cultured in Dulbecco's Modified Eagle Medium F12 supplemented with 10% heat-inactivated fetal bovine serum (FBS) and 1% penicillin/streptomycin (Pen-Strep). In some embodiments, MDA-MB-231 cells are cultured in RPMI medium supplemented with 10% FBS and 1% Pen-Strep. In some embodiments, BT-474 cells are cultured in Hybri-Care Medium 46-X prepared with 18 MΩ deionized water supplemented with 1.3 mM of sodium bicarbonate, 10% heat-inactivated FBS, and 1% Pen-Strep. Cells may be passaged on 10 cm cell culture treated dishes at 37° C./5% CO2 and passaged by washing with Dulbecco's phosphate buffered saline and then incubating with Trypsin at 37° C. with 5% CO2 for 7 min before splitting at a 1:5 ratio.

CTG (e.g., read by the CTG reader 115) is a cell viability assay that quantifies the amount of ATP present at the end of an experiment as an indicator of the number of live cells. The electronic controller 155 measures ATP content in each condition as a readout by a luminescent signal, which is normalized by the luminescence of the control (e.g., a control well) to determine cell viability relative to on-plate controls. In some embodiments, the CTG assay is performed by first removing 100 μL of media from each well on the well-plate 105, which is then replaced with an equal amount of CellTiter-Glo reagent (e.g., Promega, G7572). Assayed plates may be shaken at 500 RPM for 20 minutes and allowed to rest for 10 minutes. In some instances, 100 μL of volume from each well is transferred to a white 96-well plate (e.g., Perkin Elmer, 6005680). The electronic controller 155 collects luminescence data from each well using an Envision plate reader (e.g., Perkin Elmer) and normalized against the solvent control to measure ATP content. The electronic controller 155 fits a 4-parameter hill curve to the dose response to determine the EC₅₀ and depth of response.

The QPI microscope system 110 measures the correlation, using the electronic controller 155, between variables using the Pearson correlation coefficient as implemented in, for example, Matlab which tests the null hypothesis that there is no relationship between the variables. The QPI microscope system 110 may also determine, using the electronic controller 155, Lin's concordance coefficient to measure the concordance between variables. In some instances, the electronic controller 155 determines the confidence interval by bootstrapping based on resampling the observed data 10,000 times and reporting the confidence interval as the minimum and maximum of the middle 95% of these data.

The mixture of resistant and sensitive cells can be simulated by randomly selecting control cells to mix into the drug-treated population at mixing ratios of 50%, 25%, 10%, 5%, 2%, 1%, 0.1%, and 0.01%. The electronic controller 155 uses this mixture to identify resistant cells using threshold growth rates ranging from −0.1 h⁻¹ to 0.15 h⁻¹ and for each threshold, determines precision and recall of the identified resistant cells. The electronic controller 155 parameterizes the precision and recall measurements by threshold-specific growth rates and uses the area under the precision-recall curve (AUPRC). Generally, a higher AUPRC indicates a more effective classifier. To compare AUPRCs across mixing ratios, the electronic controller 155 compares AUPRCs to a no-skill line. In some instances, all AUPRCs reported are normalized by the area above the no skill line, via the electronic controller 155, which represents the best possible performance of the classifier at a specified mixing ratio. To reduce errors due to random sampling of the control population, especially at the lowest mixing ratios, mixing may be repeated about 100 times for each condition and the electronic controller 155 sorts the data based on a measured AUC. In some embodiments, the electronic controller 155 plots a median precision and recall value for each tested threshold and reports the median AUC.

In some examples, the electronic controller 155 median filters cell mass versus time data with a minimum length of 20 frames, with a kernel size of 5 frames, to remove small fluctuations. Data with a mean mass lower than 110 pg is removed from the analysis, as data with a mean mass lower than 110 pg may be debris. The electronic controller 155 time shifts each mass over time plot, such that the first mass measurement of each cell starts at t=0. The electronic controller 155 applies a linear regression to find the slope, which defines the growth rate (pg/h), and the y-intercept, which is used as the initial mass. The electronic controller 155 determines SGR by computing the growth rate divided by the initial mass. The electronic controller 155 uses the standard error of the estimate (sy.x) and normalized slope (specific growth rate; k) to remove outliers. An outlier may be defined as 3 median absolute deviation from the median.

In some embodiments, an overall mass of an imaging location is the summation of the mass of individual pixels after background correction. The electronic controller 155 collects overall mass for each location over time, median filtered, then normalized by the mass at the state of the imaging by the QPI microscope 120. In some embodiments, all locations in all wells for a given condition are averaged. In some instances, the electronic controller 155 determines the standard deviation for each triplicate of wells.

The electronic controller 155 fits average SGR data for individual treatments to both a response (Hill equation) and a no-response (flat line) model. The response model is a four-parameter logistic (Hill equation) function for fitting SGR versus concentration, C:

$$SGR = {E}_{max} + \frac{{E}_{0} - {E}_{max}}{{( 1 + ( C * ( {EC}_{50} ) ) )}^{HS}} \qquad Equation 2$$

Such that E₀ is asymptote at lowest concentration, Eₘₐₓ is asymptote at maximum concentration, EC₅₀ is the inflection point of the hill curve, and HS is the Hill slope. The no-response model is a flat line parallel to the concentration axis. The residual variance from each fit is compared using an F-test with a p value of 0.01. For responding conditions (logistic fit better than no-response fit as determined by F-test), depth of response (DoR) is computed as:

$$DoR = \frac{{E}_{0} - {E}_{max}}{{E}_{0}} \qquad Equation 3$$

To measure dynamic changes in SGR over time, the electronic controller 155 breaks up cell mass versus time tracks into overlapping 24 h intervals centered on each imaging time point in the experiment for a total of 144 total intervals. In some embodiments, the electronic controller 155 filters the tracks within each interval for a minimum path length of 20 frames within each interval, a minimum mean mass of 110 pg, and goodness of fit to a linear model, as described above. The electronic controller 155 determines the specific growth rate for each cell in each time interval by time shifting each track to start at t=0, and using a linear regression to find the rate of mass accumulation (e.g., based on cell mass and the period of time) and this slope was normalized by the y-intercept of each regression of the time-shifted data in the interval. In some instances, 8 adjacent intervals are binned together to produce 18 bins throughout the duration of the experiment. The electronic controller 155 determines the kernel density function in each bin using the mean SGR of each cell in the bin.

The electronic controller 155 measures the Hellinger distance between growth rate distributions by fitting a probability density function (PDF) to the distribution of raw growth rates of each cell or cluster normalized by its initial mass such that an integral over the PDF is equal to 1. The Hellinger distance may be defined as:

$${H}^{2} ( f , g ) = 1 - \int \sqrt{f ( x ) g ( x )} dx = \frac{1}{2} \int {( \sqrt{f ( x )} - \sqrt{g ( x )} )}^{2} dx \qquad Equation 4$$

which is then discretized to:

$${H}^{2} ( S , T ) = \frac{1}{2} \sum {( \sqrt{{s}_{i}} - \sqrt{{t}_{i}} )}^{2} \qquad Equation 5$$

Such that H is the computed Hellinger distance between probability distribution functions s and t. The electronic controller 155 determines the sum using a histogram bin size of 10⁻⁴ h⁻¹ such that the bin size is small enough to capture differences in growth rate for BT-47 cells, the slowest growing cell line tested.

The electronic controller 155 determines the ToR by plotting the Hellinger distance between the drug-treated group and the control against time, and fitting the data to the following equation:

$$H = a - b * {e}^{ct} \qquad Equation 6$$

Such that a, b, and c are fit parameters to minimize the sum of least squared residuals. The electronic controller 155 fits Equation 6 to the Hellinger distance measured between the controls to find the Hellinger distance threshold as the maximum distance between controls for each cell line. The electronic controller 155 determines the ToR by fitting the noted model to the Hellinger distance versus time for each therapy and analytically solving for t:

$$t = \frac{1}{c} log ( \frac{a - H}{b} ) \qquad Equation 7$$

Where H is equal to the threshold Hellinger distance, and a, b, and c are the fitting parameters to minimize the sum of least squared residuals.

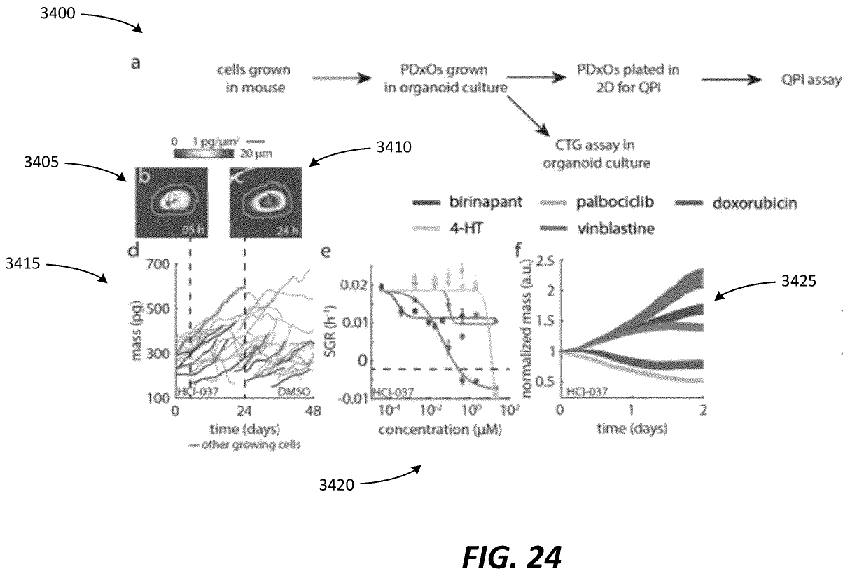

In some embodiments, the electronic controller 155 determines a treatment response for each therapy treatment sample of the plurality of treatment samples based on the plurality of response parameters. In some embodiments, the QPI microscope system 110 performs QPI on cells from patient-derived organoids and direct-from-patient samples. For example, the electronic controller 155 analyzes data on cells from a set of 5 organoids representing samples collected from two patients, as illustrated by a workflow 3400 in FIG. 24. The workflow 3400 is an experiment workflow illustrating how assays are analyzed by the QPI microscope system 110. In some embodiments, the QPI microscope system 110 images cells for 48 h, and uses an automatic segmentation algorithm, via the electronic controller 155, to identify individual cells from background, as shown in images 3405 and 3410 of FIG. 24. The image 3405 shows a PDxO cell (e.g., HCI-037) at 5 h into imaging and the image 3410 shows the PDxO cell at 24 h into imaging. The electronic controller 155 determines the mass of each cell by summing the cell mass over the segmented area. In some embodiments, plots of mass over time reveal single cell growth rates, as shown by graph 3415 of FIG. 24. The graph 3415 is a mass vs. time plot showing DMSO treated cells growing cells at a typical location. Cells outlined in green are shown as green on the plot, other growing cells are shown in purple. The electronic controller 155 determine the specific growth rate (SGR) by fitting a line to time-series mass measurements and normalizing by an initial mass predicted by the fit to account for the effect of cell size on growth rate. The electronic controller 155 fits SGR as a function of dose to a sigmoidal dose response curve using a 3-parameter Hill model to extract dose response measurements, as shown by graph 3420 of FIG. 24. The graph 3420 is a dose response plot showing strong response of HCI-037 to 4-HT and doxorubicin, but a lesser response to vinblastine. In some embodiments, EC₅₀ measured by the QPI microscope system 110 is strongly concordant with gold standard techniques such as CTG over 27 conditions (e.g., 9 compounds, 3 breast cancer cell lines). A plot of normalized mass vs. time shows the population average temporal growth response at EC₅₀. As with cell line data, QPI data on cells from PDxO resolves differences in temporal dynamics of response to therapy, as shown in graph 3425 of FIG. 24. The graph 3425 includes normalized mass shows the temporal dynamics of HCI-037 response to a panel of therapies at the EC₅₀, showing a drug dependent response. In some embodiments, the electronic controller 155 is configured to determine a drug response of each of the plurality of treatment samples from patient derived organoids.

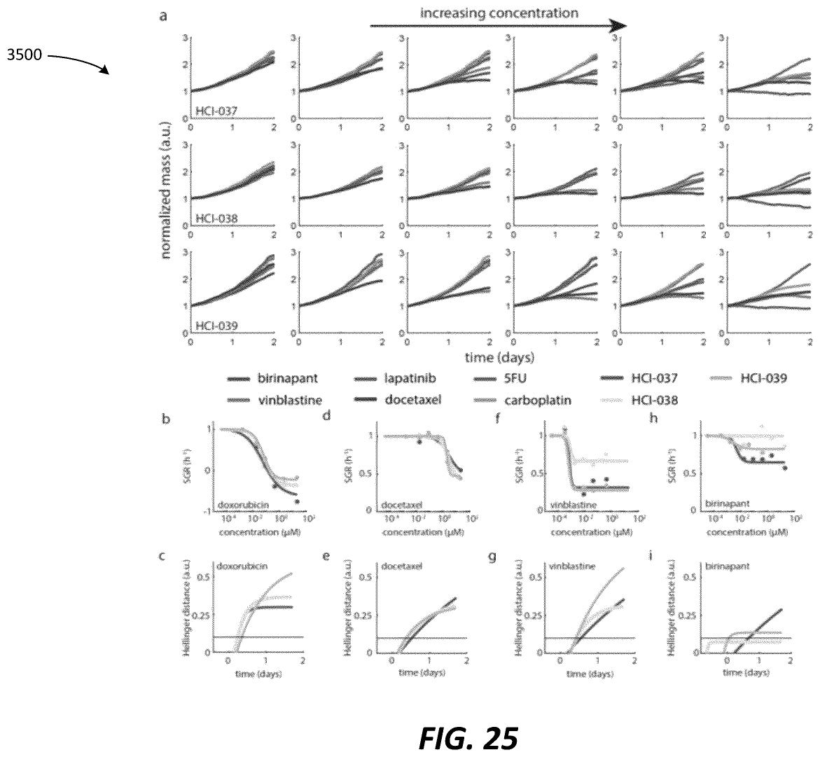

In some embodiments, data from a set of samples shows that the QPI microscope system 110 differentiates response based on site of origin, as shown by the graphs 3500 of FIG. 25. The graphs 3500 includes graph A of the graphs 3500 showing normalized mass vs. time for HCI-037 (primary), HCI-038 (metastatic), and HCI-039 (pleural effusion) showing reduction in growth as concentration increases. Graphs B, C, D, and E of the graphs 3500 show dose response plots showing the differential response between different disease models. Graphs F, G, H, and I of the graphs 3500 show Hellinger distance vs. time plots showing the time of response for the different models treated with various therapies. In some embodiments, HCI-037, HCI-038, and HCI-039, PDxO models are used that are derived from primary lesion, bone metastasis, and pleural effusion, respectively, at different time points from a single patient during the course of treatment and progressive disease. In some embodiments, the electronic controller 155 is configured to determine a difference drug response from patient derived organoids. In some embodiments, the patient derived organoids are tumor organoids.

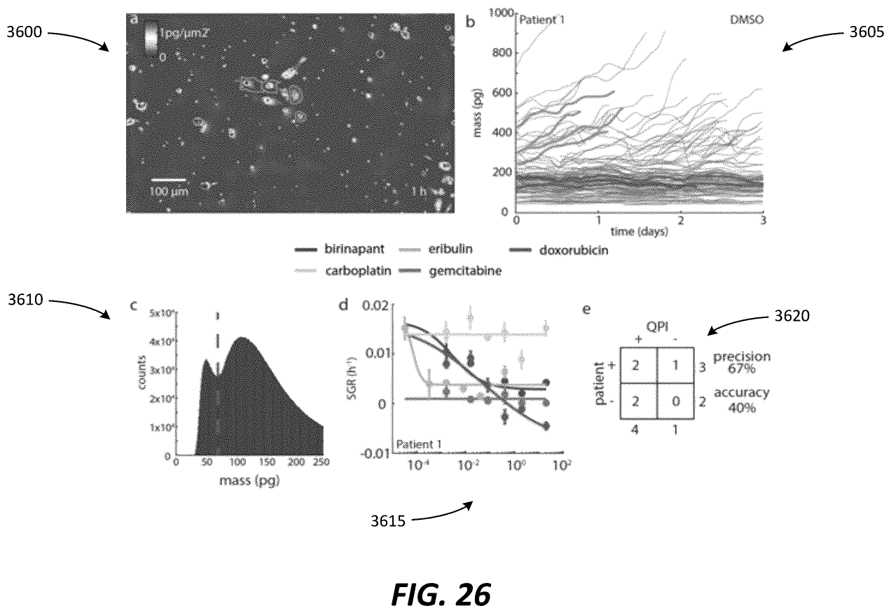

While all three PDxO models demonstrate a similar response profile to doxorubicin and docetaxel, the PDxO models did not demonstrate the same response across the entire set of therapies. For example, more progressive disease models (e.g., bone metastasis and pleural effusion) show a stronger response to vinblastine than expected based on data from the primary lesion. For birinapant, while the EC₅₀ is similar for HCI-037 and HCI-039, HCI-038 had no response indicating this model exhibited resistant. Furthermore, even though the EC₅₀ for HCI-037 and HCI-039 is similar, HCI-037 has a much greater depth of response and that HCI-039 has a much faster time of response indicating a differential growth response behavior between the different disease models. Together, the QPI microscope system 110 interrogates the differential drug response between different patient derived models from the same patient, which may be useful for detecting the emergence of resistance. In some embodiments, the QPI microscope system 110 applies multiparametric QPI to primary cells from two clinical samples stored in cell bank at, for example, the Huntsman Cancer Institute plated after thawing from liquid nitrogen and CD45+ depleted to reduce the number of immune cells, as shown in FIG. 26.

For example, the primary cells are plated as a 2D monolayer in Clever's complete medium in a 96 well plate (e.g., the well-plate 105) and are allowed to acclimate to cell culture conditions for 18 h prior to dosing with treatments assigned to each patient by a physician. Even after CD45+ depletion, there are still small cells that maintain mass throughout the duration of imaging as shown by the image 3600 of FIG. 26; however, the cells do not grow as robustly as tumor cells. The image 3600 is a QPI image of primary cells from a direct from thaw patient derived sample. A graph 3605 of FIG. 26 shows a mass vs. time plot for cells shown in the image 3600. By plotting a histogram of the mass of all cells measured during the experiment (e.g., from Patient #1), there exists a bimodal distribution, such that one peak represents low mass cells and the other represents high mass cells, as shown in graph 3610 of FIG. 26. The graph 3610 is a histogram of cell mass used to computationally filter, using the electronic controller 155, for low mass cells (shown in red in the image 3600 and the graph 3605). By using a cutoff between the peaks at, for example, 72 pg, low mass cells can be isolated that are not growing and remove such cells from analysis. Thus, higher mass tumor cells may be studied, without the results being contaminated by the other cells in the population. In some embodiments, the electronic controller 155 is configured to determine a tumor cell from a non-tumor cell in the therapy treatment sample based on the results prior to a response assay using QPI. Using this cutoff, the electronic controller 155 measures the dose response for cells in each condition to determine the EC₅₀ and DoR for each condition studied for the primary cells, as shown in the graph 3615. The graph 3615 is a dose response plot for primary cells treated with therapies assigned to the patient by a physician. In some embodiments, comparing the QPI dose results to an outcome of the patient shows weak agreement with a measured accuracy of 40% and precision of 67%, as shown in the graph 3620 of FIG. 26. The graph 3620 shows Hellinger distance vs. time plots for primary cells treated with cancer therapy used to measure the time of response by the electronic controller 155. For Patient #2, similar heterogeneity in size may occur between big cells that grew robustly and small cells that did not grow much. In some instances, the population does not display a bimodal distribution like Patient #1 making the use of a simple cutoff impractical to differentiate the two populations. In some embodiments, the electronic controller 155 determines a drug response in cells from patient derived samples. For example, the electronic controller 155 determines the drug response in therapy treatment samples from patient derived samples.

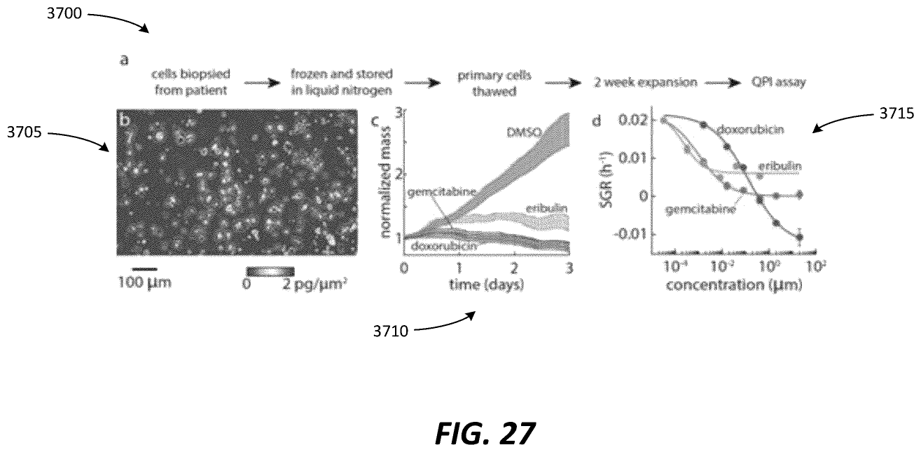

Without separating the two populations, the fitting code for EC₅₀ and DoR fails to find a response. In some embodiments, both samples are expanded from primary cells in 2D culture using optimized ex vivo culture conditions for 2 weeks, changing media every 3 days to further purify the tumor cells, as shown in a workflow 3700 of FIG. 27. The workflow 3700 shows how cells are handled from biopsy from patient through to the drug response assay using the QPI microscope system 110.

After short-term expansion, the sample is enriched for high mass tumor cells relative to the high content of low mass immune cells in the direct from thaw primary sample, as shown in the image 3705 of FIG. 27. The image 3705 shows a QPI image of primary cells treated with DMSO. For Patient #1, normalized mass over time shows that, on average, DMSO treated cells grow robustly throughout the duration of the experiment whereas cells treated with drug at the concentration just above the EC₅₀ show significant response to therapy. In some embodiments, cells treated with eribulin show a lesser overall response relative to cells treated with doxorubicin or gemcitabine, which is consistent with dose response plots showing eribulin with a lower DoR relative to the other therapies tested, as shown by graphs 3710 and 3715 of FIG. 27. The graph 3710 of FIG. 27 shows a plot of normalized mass over time showing average of all cells tested in DMSO control and at EC₅₀ for each condition. The graph 315 of FIG. 27 shows a dose response plot showing average SGR vs. concentration for each therapy tested. The normalized mass vs. time also shows that tumor cells exposed to eribulin grow at a similar rate as the control for the first day (paired t-test, p=0.002) before the growth rate declines to approximately 0.0012 h−1 for the rest of the experiment. Together, this indicates that eribulin causes a lesser response from the tumor cells than either gemcitabine or doxorubicin, which both had a lower EC₅₀ than eribulin.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein. No functional language used in claims appended herein is to be construed as invoking 35 U.S.C. § 112 (f) interpretations as “means-plus-function” language unless specifically expressed as such by use of the words “means for” or “steps for” within the respective claim.

When introducing elements of the present invention or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. The term “exemplary” is not intended to be construed as a superlative example but merely one of many possible examples.

What is claimed is:

1. A microscope imaging system comprising:
a quantitative phase imaging microscope including
a stage configured to hold and move a well plate having a plurality of therapy treatment samples;
an array of light emitting diodes (LEDs) configured to illuminate the plurality of therapy treatment samples;
a camera configured to capture images of the plurality of therapy treatment samples over a period of time using an amount of illumination provided by the array of LEDs; and
an electronic controller communicatively coupled to the quantitative phase imaging microscope, the electronic controller configured to:
receive a plurality of images of the plurality of therapy treatment samples over the period of time from the camera;
determine a cell mass for each therapy treatment sample of the plurality of therapy treatment samples based on each image of the plurality of images;
track the cell mass over the period of time for each therapy treatment sample of the plurality of therapy treatment samples;
determine a plurality of response parameters for each therapy treatment sample of the plurality of therapy treatment samples based on the cell mass over the period of time; and
determine a treatment response for each therapy treatment sample of the plurality of therapy treatment samples based on the plurality of response parameters.

2. The microscope imaging system of claim 1, wherein the plurality of response parameters include at least one selected from the group consisting of a specific growth rate, a half maximal effective concentration, a depth of response, a time of response, and a standard deviation of response.

3. The microscope imaging system of claim 1, wherein each therapy treatment sample of the plurality of therapy treatment samples includes live cancer cells exposed to a different therapeutic drug.

4. The microscope imaging system of claim 3, wherein the cell mass is a mass of the live cancer cells exposed to the different therapeutic drugs.

5. The microscope imaging system of claim 1, wherein the quantitative phase imaging microscope further includes:
a lens configured to focus the camera on the plurality of therapy treatment samples.

6. The microscope imaging system of claim 1, wherein when determining the cell mass for each therapy treatment sample of the plurality of therapy treatment samples, the electronic controller is further configured to:
determine a phase shift of the amount of illumination passing through each therapy treatment sample of the plurality of therapy treatment samples.

7. The microscope imaging system of claim 1, wherein when tracking the cell mass over the period of time for each therapy treatment sample of the plurality of therapy treatment samples, the electronic controller is further configured to:
determine a mass accumulation rate for each therapy treatment sample of the plurality of therapy treatment samples based on the determined cell mass and the period of time.

8. A method for determining treatment response parameters with a microscope imaging system, the method comprising:
receiving, by an electronic controller, a plurality of images of a plurality of therapy treatment samples over a period of time from a camera;
determining, via the electronic controller, a cell mass for each therapy treatment sample of the plurality of therapy treatment samples based on each image of the plurality of images;
tracking, via the electronic controller, the cell mass over the period of time for each therapy treatment sample of the plurality of therapy treatment samples; and
determining, via the electronic controller, a plurality of response parameters for each therapy treatment sample of the plurality of therapy treatment samples based on the cell mass over the period of time.

9. The method of claim 8, further comprising:
determining, via the electronic controller, a treatment response for each therapy treatment sample of the plurality of therapy treatment samples based on the plurality of response parameters.

10. The method of claim 8, wherein the plurality of response parameters include at least one selected from the group consisting of a specific growth rate, a half maximal effective concentration, a depth of response, a time of response, and a standard deviation of response.

11. The method of claim 8, wherein each therapy treatment sample of the plurality of therapy treatment samples includes live cancer cells exposed to a different therapeutic drug.

12. The method of claim 11, wherein the cell mass is a mass of the live cancer cells exposed to the different therapeutic drugs.

13. The method of claim 8, further comprising:
determining, via the electronic controller, a phase shift of the amount of illumination passing through each therapy treatment sample of the plurality of therapy treatment samples.

14. The method of claim 8, further comprising:
determining, via the electronic controller, a mass accumulation rate for each therapy treatment sample of the plurality of therapy treatment samples based on the determined cell mass and the period of time.

15. A quantitative phase imaging microscope comprising:
a camera configured to capture images of a plurality of therapy treatment samples over a period of time; and
an electronic controller communicatively coupled to the quantitative phase imaging microscope, the electronic controller configured to:
receive a plurality of images of the plurality of therapy treatment samples over the period of time from the camera;
determine a cell mass for each therapy treatment sample of the plurality of therapy treatment samples based on each image of the plurality of images;
track the cell mass over the period of time for each therapy treatment sample of the plurality of therapy treatment samples; and
determine a plurality of response parameters for each therapy treatment sample of the plurality of therapy treatment samples based on the cell mass over the period of time.

16. The quantitative phase imaging microscope of claim 15, wherein the electronic controller is configured to:
determine a treatment response for each therapy treatment sample of the plurality of therapy treatment samples based on the plurality of response parameters.

17. The quantitative phase imaging microscope of claim 15, wherein the electronic controller is configured to:
determine the plurality of response parameters for each therapy treatment sample of the plurality of therapy treatment samples in cells from a patient derived organoid.

18. The quantitative phase imaging microscope of claim 15, wherein the electronic controller is configured to:
determine a difference in the plurality of response parameters between each therapy treatment sample of the plurality of treatment samples.

19. The quantitative phase imaging microscope of claim 18, wherein the difference in the plurality of response parameters is determined from a tumor organoid.

20. The quantitative phase imaging microscope of claim 15, wherein the electronic controller is configured to:
determine a tumor cell from a non-tumor cell for each therapy treatment sample of the plurality of therapy treatment samples based on the cell mass over the period of time.
